(19) United States
(12) Patent Application Publication
(10) Pub. No.: US 2005/0277122 A1
(43) Pub. Date: Dec. 15, 2005

(54) Devices and methods for contacting fluid with a chemical array

(76) Inventor: Joseph P. Fredrick, Palo Alto, CA (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 10/868,330

(22) Filed: Jun. 14, 2004

(51) Int. Cl. C12Q 1/68; C12M 1/34

(52) U.S. Cl. 435/6; 435/287.2

(57) ABSTRACT

The subject invention provides devices and methods for contacting a fluid with a chemical array. Device embodiments include a reservoir plate that includes a support having a plurality of holes therethrough alignable with a chemical array when the reservoir plate and chemical array are operatively positioned relative to each other. Method for using the subject reservoir plates are also provided, as well as systems and kits.

### BACKGROUND OF THE INVENTION

Chemical arrays such as biopolymer arrays (for example polynucleotide array such as DNA or RNA arrays), are known and are used, for example, as diagnostic or screening tools. Such arrays include regions of usually different sequence polynucleotides arranged in a predetermined configuration on a substrate. These regions (sometimes referenced as “features”) are positioned at respective locations (“addresses”) on the substrate. The arrays, when exposed to a sample, will exhibit an observed binding pattern. This binding pattern can be detected upon interrogating the array. For example all polynucleotide targets (for example, DNA) in the sample can be labeled with a suitable label (such as a fluorescent compound), and the fluorescence pattern on the array accurately observed following exposure to the sample. Assuming that the different sequence polynucleotides were correctly deposited in accordance with the predetermined configuration, then the observed binding pattern will be indicative of the presence and/or concentration of one or more polynucleotide components of the sample.

Arrays can be fabricated by depositing previously obtained biopolymers onto a substrate, or by in situ synthesis methods. The in situ fabrication methods include those described in U.S. Pat. No. 5,449,754 for synthesizing peptide arrays, and in U.S. Pat. No. 6,180,351 and WO 98/41531 and the references cited therein for synthesizing polynucleotide arrays. Further details of fabricating biopolymer arrays are described in U.S. Pat. No. 6,242,266, U.S. Pat. No. 6,232,072, U.S. Pat. No. 6,180,351, and U.S. Pat. No. 6,171,797. Other techniques for fabricating biopolymer arrays include known light directed synthesis techniques. Methods for sample preparation, labeling, and hybridizing are disclosed for example in U.S. Pat. No. 6,201,112, U.S. Pat. No. 6,132,997, U.S. Pat. No. 6,235,483, and U.S. patent publication 20020192650.

In array fabrication, the probes formed at each feature usually are expensive. Additionally, sample quantities available for testing are usually also very small and it is therefore desirable to simultaneously test the same sample against a large number of different probes on an array. These conditions make it desirable to produce arrays with large numbers of very small (for example, in the range of tens or one or two hundred microns diameter), closely spaced features (for example many thousands of features). After an array has been exposed to a sample, the array is read with a reading apparatus (such as an array “scanner”) which detects the signals (such as a fluorescence pattern) from the array features. Such a reader should typically have a very fine resolution (for example, in the range of one to 100 microns). The signal image resulting from reading the array can then be digitally processed to evaluate which regions (pixels) of read data belong to a given feature as well as the total signal strength from each of the features. The foregoing steps, separately or collectively, are referred to as “feature extraction”.

Handling fluids for contacting with a chemical array, e.g., either for a binding assay (such as a hybridization assay), washing step, and the like, is of great importance to array assays, e.g., particularly in view of the oftentimes rare and expensive fluids employed such as target assay fluids and the like. Conventional fluid handling is often accomplished manually. However, manual fluid handling is time consuming and labor intensive.

### SUMMARY OF THE INVENTION

The subject invention provides devices and methods for contacting with a defined region of an array carrier carrying at least one chemical array. Embodiments of the subject devices include a reservoir plate that includes a support having a plurality of holes therethrough alignable with a chemical array when the reservoir plate and chemical array are operatively positioned relative to each other. In certain embodiments, a reservoir plate is configured to be used with a set of chemical arrays present on a common carrier, where the common carrier may be an array holder holding one or more chemical arrays or a one-piece substrate having a surface upon which one or more chemical arrays are disposed. The subject invention also includes methods for contacting a fluid with a defined region of a surface of an array carrier where embodiments include positioning a reservoir plate relative to the surface of the carrier and introducing a fluid through at least one hole of the reservoir plate such that the fluid contacts a defined region of the carrier. Systems and kits are also provided.

### BRIEF DESCRIPTION OF THE DRAWINGS

FIG. 1 shows an embodiment of an array assembly.

FIG. 2 shows an enlarged view of a portion of FIG. 1 showing spots or features.

FIG. 3 is an enlarged view of a portion of the substrate of FIG. 2.

FIG. 4 shows an embodiment of an array assembly in the form of a set of chemical arrays held together by a common carrier in the form of a one-piece substrate on which the arrays are disposed.

FIG. 5 is a view similar to FIG. 4 but illustrating an alternate embodiment of the apparatus.

FIG. 6 is a cross-section along the line 5-5 of FIG. 5.

FIG. 7 is a top view of separated sub-sets of arrays from the array assembly of FIG. 4 or 6, each separated sub-set carried on a separate substrate section.

FIGS. 8A, 8B and 8C show top views of embodiments of reservoir plates according to the subject invention.

FIG. 9 shows a partial view of an embodiment of a reservoir plate according to the subject invention.

FIG. 10 shows a surface 311a of the reservoir plate portion shown in FIG. 9.

FIG. 11 shows a partial view of an embodiment of a reservoir plate operatively positioned relative to an array assembly to provide a fluid contacting structure according to the subject invention.

FIGS. 12A and 12B show the relative positioning of an embodiment of a reservoir plate with an array assembly to provide a fluid contacting structure.

FIGS. 13A and 13B show the relative positioning of an embodiment of a reservoir plate with an array assembly to provide a fluid contacting structure and

FIG. 13C shows a cross section through the so provided fluid contacting structure showing fluid contacted with arrays through the reservoir plate.

FIGS. 14A and 14B show an embodiment of fluid removal and flushing through the reservoir plate according to the subject invention.

FIGS. 15A, 15B and 15C show another embodiment of fluid removal and flushing according to the subject invention.

FIG. 16 shows an exemplary embodiment of an automated system that may be employed in the practice of the subject invention.

To facilitate understanding, identical reference numerals have been used, where practical, to designate the same elements which are common to different figures. Drawings are not necessarily to scale. Throughout this application any different members of a generic class may have the same reference number followed by different letters (for example, arrays 12a, 12b, 12c, and 12d may generically be referenced as “arrays 12”)

### DEFINITIONS

Throughout the present application, unless a contrary intention appears, the following terms refer to the indicated characteristics.

A “biopolymer” is a polymer of one or more types of repeating units. Biopolymers are typically found in biological systems and particularly include polysaccharides (such as carbohydrates), and peptides (which term is used to include polypeptides, and proteins whether or not attached to a polysaccharide) and polynucleotides as well as their analogs such as those compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. Specifically, a “biopolymer” includes DNA (including cDNA), RNA and oligonucleotides, regardless of the source.

A “biomonomer” references a single unit, which can be linked with the same or other biomonomers to form a biopolymer (for example, a single amino acid or nucleotide with two linking groups one or both of which may have removable protecting groups). A biomonomer fluid or biopolymer fluid reference a liquid containing either a biomonomer or biopolymer, respectively (typically in solution).

A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides.

An “oligonucleotide” generally refers to a nucleotide multimer of about 10 to 100 nucleotides in length, while a “polynucleotide” includes a nucleotide multimer having any number of nucleotides.

A chemical “array”, unless a contrary intention appears, includes any one, two or three-dimensional arrangement of addressable regions bearing a particular chemical moiety or moieties (for example, biopolymers such as polynucleotide sequences) associated with that region. For example, each region may extend into a third dimension in the case where the substrate is porous while not having any substantial third dimension measurement (thickness) in the case where the substrate is non-porous. An array is “addressable” in that it has multiple regions (sometimes referenced as “features” or “spots” of the array) of different moieties (for example, different polynucleotide sequences) such that a region at a particular predetermined location (an “address”) on the array will detect a particular target or class of targets (although a feature may incidentally detect non-targets of that feature). An array feature is generally homogenous in composition and concentration and the features may be separated by intervening spaces (although arrays without such separation can be fabricated). In the case of an array, the “target” will be referenced as a moiety in a mobile phase (typically fluid), to be detected by probes (“target probes”) which are bound to the substrate at the various regions. However, either of the “target” or “target probes” may be the one which is to be detected by the other (thus, either one could be an unknown mixture of polynucleotides to be detected by binding with the other).

An “array layout” or “array characteristics”, refers to one or more physical, chemical or biological characteristics of the array, such as positioning of some or all the features within the array and on a substrate, one or more feature dimensions, or some indication of an identity or function (for example, chemical or biological) of a moiety at a given location, or how the array should be handled (for example, conditions under which the array is exposed to a sample, or array reading specifications or controls following sample exposure).

“Hybridizing” and “binding”, with respect to polynucleotides, are used interchangeably.

A “plastic” is any synthetic organic polymer of high molecular weight (for example at least 1,000 grams/mole, or even at least 10,000 or 100,000 grams/mole.

“Flexible” with reference to a substrate or substrate web (including a housing or one or more housing component such as a housing base and/or cover), references that the substrate can be bent 180 degrees around a roller of less than 1.25 cm in radius. The substrate can be so bent and straightened repeatedly in either direction at least 100 times without failure (for example, cracking) or plastic deformation. This bending must be within the elastic limits of the material. The foregoing test for flexibility is performed at a temperature of 20° C. “Rigid” refers to a substrate (including a housing or one or more housing component such as a housing base and/or cover) which is not flexible, and is constructed such that a segment about 2.5 by 7.5 cm retains its shape and cannot be bent along any direction more than 60 degrees (and often not more than 40, 20, 10, or 5 degrees) without breaking.

A “web” references a long continuous piece of substrate material having a length greater than a width. For example, the web length to width ratio may be at least 5/1, 10/1, 50/1, 100/1, 200/1, or 500/1, or even at least 1000/1.

When one item is indicated as being “remote” from another, this is referenced that the two items are at least in different buildings, and may be at least one mile, ten miles, or at least one hundred miles apart. When different items are indicated as being “local” to each other they are not remote from one another (for example, they can be in the same building or the same room of a building). “Communicating”, “transmitting” and the like, of information reference conveying data representing information as electrical or optical signals over a suitable communication channel (for example, a private or public network, wired, optical fiber, wireless radio or satellite, or otherwise). Any communication or transmission can be between devices which are local or remote from one another. “Forwarding” an item refers to any means of getting that item from one location to the next, whether by physically transporting that item or using other known methods (where that is possible) and includes, at least in the case of data, physically transporting a medium carrying the data or communicating the data over a communication channel (including electrical, optical, or wireless). “Receiving” something means it is obtained by any possible means, such as delivery of a physical item (for example, an array or array carrying package). When information is received it may be obtained as data as a result of a transmission (such as by electrical or optical signals over any communication channel of a type mentioned herein), or it may be obtained as electrical or optical signals from reading some other medium (such as a magnetic, optical, or solid state storage device) carrying the information. However, when information is received from a communication it is received as a result of a transmission of that information from elsewhere (local or remote).

When two items are “associated” with one another they are provided in such a way that it is apparent one is related to the other such as where one references the other. For example, an array identifier can be associated with an array by being on the array assembly (such as on the substrate or a housing) that carries the array or on or in a package or kit carrying the array assembly. Items of data are “linked” to one another in a memory when a same data input (for example, filename or directory name or search term) retrieves those items (in a same file or not) or an input of one or more of the linked items retrieves one or more of the others. In particular, when an array layout is “linked” with an identifier for that array, then an input of the identifier into a processor which accesses a memory carrying the linked array layout retrieves the array layout for that array.

A “computer”, “processor” or “processing unit” are used interchangeably and each references any hardware or hardware/software combination which can control components as required to execute recited steps. For example a computer, processor, or processor unit includes a general purpose digital microprocessor suitably programmed to perform all of the steps required of it, or any hardware or hardware/software combination which will perform those or equivalent steps. Programming may be accomplished, for example, from a computer readable medium carrying necessary program code (such as a portable storage medium) or by communication from a remote location (such as through a communication channel).

A “memory” or “memory unit” refers to any device which can store information for retrieval as signals by a processor, and may include magnetic or optical devices (such as a hard disk, floppy disk, CD, or DVD), or solid state memory devices (such as volatile or non-volatile RAM). A memory or memory unit may have more than one physical memory device of the same or different types (for example, a memory may have multiple memory devices such as multiple hard drives or multiple solid state memory devices or some combination of hard drives and solid state memory devices).

An array “assembly” includes a substrate and at least one chemical array on a surface thereof. Assemblies include arrays on a one-piece substrate or different substrate sections. An assembly may include other features (such as a housing with a chamber from which the substrate sections can be removed). “Array unit” may be used interchangeably with “array assembly”.

“Reading” signal data from an array refers to the detection of the signal data (such as by a detector) from the array. This data may be saved in a memory (whether for relatively short or longer terms).

A “package” is one or more items (such as an array assembly optionally with other items) all held together (such as by a common wrapping or protective cover or binding). Normally the common wrapping will also be a protective cover (such as a common wrapping or box) which will provide additional protection to items contained in the package from exposure to the external environment. In the case of just a single array assembly a package may be that array assembly with some protective covering over the array assembly (which protective cover may or may not be an additional part of the array unit itself).

It will also be appreciated that throughout the present application, that words such as “cover”, “base” “front”, “back”, “top”, “upper”, and “lower” are used in a relative sense only.

“May” refers to optionally.

When two or more items (for example, elements or processes) are referenced by an alternative “or”, this indicates that either could be present separately or any combination of them could be present together except where the presence of one necessarily excludes the other or others.

The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

A “stringent hybridization” and “stringent hybridization wash conditions” in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO4, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions that set forth the conditions which determine whether a nucleic acid is specifically hybridized to a surface bound nucleic acid. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

Any recited method can be carried out in the order of events recited or in any other order which is logically possible. Reference to a singular item, includes the possibility that there are plural of the same item present. All patents and other references cited in this application, are incorporated into this application by reference except insofar as anything in those patents or references, including definitions, conflicts with anything in the present application (in which case the present application is to prevail).

### DETAILED DESCRIPTION OF EMBODIMENTS OF THE INVENTION

The subject invention provides devices and methods for contacting with a defined region of an array carrier carrying at least one chemical array. Embodiments of the subject devices include a reservoir plate that includes a support having a plurality of holes therethrough alignable with a chemical array when the reservoir plate and chemical array are operatively positioned relative to each other. In certain embodiments, a reservoir plate is configured to be used with a set of chemical arrays present on a common carrier, where the common carrier may be an array holder holding one or more chemical arrays or a one-piece substrate having a surface upon which one or more chemical arrays are disposed. The subject invention also includes methods for contacting a fluid with a defined region of a surface of an array carrier where embodiments include positioning a reservoir plate relative to the surface of the carrier and introducing a fluid through at least one hole of the reservoir plate such that the fluid contacts a defined region of the carrier. Systems and kits are also provided.

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the particular embodiments of methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention.

The figures shown herein are not necessarily drawn to scale, with some components and features being exaggerated for clarity.

In further describing the subject invention, chemical array embodiments are described first to provide a proper foundation for further describing the subject devices and methods for use with chemical arrays. Next, embodiments of the subject devices are described, followed by a description of the subject methods.

Chemical Arrays

As noted above, the subject invention finds use with chemical arrays and are particularly well-suited for use with multiple chemical arrays associated with the same carrier, e.g., an array set associated with a common carrier, as will be described in greater detail below. Chemical arrays find use in a variety of applications, including gene expression analysis, drug screening, nucleic acid sequencing, mutation analysis, and the like. Chemical arrays include a plurality of ligands or molecules or probes (i.e., binding agents or members of a binding pair) deposited onto the surface of a substrate in the form of an “array” or pattern.

Chemical arrays include at least two distinct polymers that differ by monomeric sequence attached to different and known locations on a carrier (substrate) surface. Each distinct polymeric sequence of the array is typically present as a composition of multiple copies of the polymer on a substrate surface, e.g., as a spot or feature on the surface of the substrate. The number of distinct polymeric sequences, and hence spots or similar structures, present on the array may vary, where a typical array may contain more than about ten, more than about one hundred, more than about one thousand, more than about ten thousand or even more than about one hundred thousand features in an area of less than about 20 cm² or even less than about 10 cm². For example, features may have widths (that is, diameter, for a round spot) in the range from about 10 μm to about 1.0 cm. In other embodiments, each feature may have a width in the range from about 1.0 μm to about 1.0 mm, usually from about 5.0 μm to about 500 μm and more usually from about 10 μm to about 200 μm. Non-round features may have area ranges equivalent to that of circular features with the foregoing width (diameter) ranges. At least some, or all, of the features are of different compositions (for example, when any repeats of each feature composition are excluded, the remaining features may account for at least about 5%, 10% or 20% of the total number of features). Interfeature areas will typically (but not essentially) be present which do not carry any polynucleotide (or other biopolymer or chemical moiety of a type of which the features are composed). Such interfeature areas may be present, for example, where the arrays are formed by processes involving drop deposition of reagents, but may not be present when, for example, photolithographic array fabrication process are used. It will be appreciated though, that the interfeature areas, when present, could be of various sizes and configurations. The spots or features of distinct polymers present on the array surface are generally present as a pattern, where the pattern may be in the form of organized rows and columns of spots, e.g. a grid of spots, across the substrate surface, a series of curvilinear rows across the substrate surface, e.g. a series of concentric circles or semi-circles of spots, and the like.

In the broadest sense, the chemical arrays are arrays of polymeric or biopolymeric ligands or molecules, i.e., binding agents, where the polymeric binding agents may be any of: peptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like.

The arrays may be produced using any convenient protocol. Various methods for forming arrays from pre-formed probes, or methods for generating the array using synthesis techniques to produce the probes in situ, are generally known in the art. See, for example, Southern, U.S. Pat. No. 5,700,637; Pirrung, et al., U.S. Pat. No. 5,143,854 and Fodor, et al. (1991) Science 251:767-777, the disclosures of which are incorporated herein by reference and PCT International Publication No. WO 92/10092. For example, probes can either be synthesized directly on the solid support or substrate to be used in the array assay or attached to the substrate after they are made. Arrays may be fabricated using drop deposition from pulse jets of either polynucleotide precursor units (such as monomers) in the case of in situ fabrication, or the previously obtained polynucleotide. Such methods are described in detail in, for example, the previously cited references including U.S. Pat. Nos. 6,242,266, 6,232,072, 6,180,351, 6,171,797, and 6,323,043; and U.S. patent application Ser. No. 09/302,898 filed Apr. 30, 1999 by Caren et al., and the references cited therein, the disclosures of which are herein incorporated by reference. Other drop deposition methods may be used for fabrication. Also, instead of drop deposition methods, photolithographic array fabrication methods may be used such as described in U.S. Pat. Nos. 5,599,695, 5,753,788, and 6,329,143, the disclosures of which are herein incorporated by reference. As mentioned above, interfeature areas need not be present, particularly when the arrays are made by photolithographic methods as described in those patents.

A variety of solid supports which in certain embodiments are referred to as carriers may be used, upon which one or more chemical arrays may be positioned. In certain embodiments, a plurality of arrays may be stably associated with one substrate. For example, a plurality of arrays may be stably associated with one substrate, where the arrays are spatially separated from some or all of the other arrays associated with the substrate.

The substrates upon which the arrays may be disposed may be selected from a wide variety of materials including, but not limited to, natural polymeric materials, particularly cellulosic materials and materials derived from cellulose, such as fiber containing papers, e.g., filter paper, chromatographic paper, etc., synthetic or modified naturally occurring polymers, such as nitrocellulose, cellulose acetate, poly (vinyl chloride), polyamides, polyacrylamide, polyacrylate, polymethacrylate, polyesters, polyolefins, polyethylene, polytetrafluoro-ethylene, polypropylene, poly (4-methylbutene), polystyrene, poly(ethylene terephthalate), nylon, poly(vinyl butyrate), cross linked dextran, agarose, etc.; either used by themselves or in conjunction with other materials; fused silica (e.g., glass), bioglass, silicon chips, ceramics, metals, and the like. For example, substrates may include polystyrene, to which short oligophosphodiesters, e.g., oligonucleotides ranging from about 5 to about 50 nucleotides in length, may readily be covalently attached (Letsinger et al. (1975) Nucl. Acids Res. 2:773-786), as well as polyacrylamide (Gait et al. (1982) Nucl. Acids Res. 10:6243-6254), silica (Caruthers et al. (1980) Tetrahedron Letters 21:719-722), and controlled-pore glass (Sproat et al. (1983) Tetrahedron Letters 24:5771-5774). Additionally, the substrate can be hydrophilic or capable of being rendered hydrophilic.

Suitable substrates may exist, for example, as sheets, tubing, spheres, containers, pads, slices, films, plates, slides, strips, disks, etc. The substrate is usually flat, but may take on alternative surface configurations. The substrate can be a flat glass substrate, such as a conventional microscope glass slide, a cover slip and the like. Common substrates used for the arrays of probes are surface-derivatized glass or silica, or polymer membrane surfaces, as described in Maskos, U. et al., Nucleic Acids Res, 1992, 20:1679-84 and Southern, E. M. et al., Nucleic acids Res, 1994, 22:1368-73.

Each array 12 may cover an area of less than 200 mm², 100 mm², or less than 50 mm², 20 mm², or less than 10 mm². Arrays 12 may be spaced apart from one another by a distance at least two, three, or four times the average distance between features within the arrays. In many embodiments, the substrate carrying the one or more arrays will be shaped generally as a rectangular solid (although other shapes are possible), having a length of more than about 4 mm and less than about 1 m, usually more than about 4 mm and less than about 600 mm, more usually less than about 400 mm; a width of more than about 4 mm and less than about 1 m, usually less than about 500 mm and more usually less than about 400 mm; and a thickness of more than about 0.01 mm and less than about 5.0 mm, usually more than about 0.1 mm and less than about 2 mm and more usually more than about 0.2 and less than about 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, the substrate may transmit at least about 20%, or about 50% (or even at least about 70%, 90%, or 95%), of the illuminating light incident on the substrate as may be measured across the entire integrated spectrum of such illuminating light or alternatively at 532 nm or 633 nm.

Immobilization of the probe to a suitable substrate may be performed using conventional techniques. See, e.g., Letsinger et al. (1975) Nucl. Acids Res. 2:773-786; Pease, A. C. et al., Proc. Nat. Acad. Sci. USA, 1994, 91:5022-5026, and AOligonucleotide Synthesis, a Practical Approach,” Gait, M. J. (ed.), Oxford, England: IRL Press (1984). The surface of a substrate may be treated with an organosilane coupling agent to functionalize the surface. See, e.g., Arkins, A Silane Coupling Agent Chemistry,” Petrarch Systems Register and Review, Eds. Anderson et al. (1987) and U.S. Pat. No. 6,258,454.

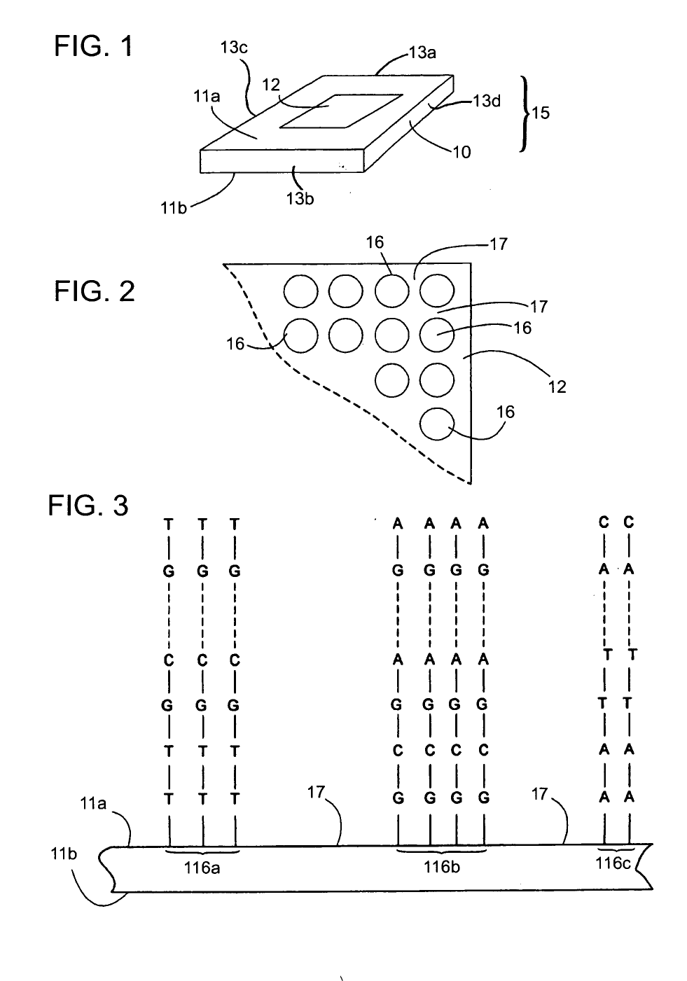

FIGS. 1-3 show an embodiment of an array assembly 15 that includes a contiguous planar substrate 10 carrying an array 12 disposed on a front surface 11a of substrate 10. Substrate 10 may also be characterized as a carrier of one or more chemical arrays. It will be appreciated though, that more than one array (any of which are the same or different) may be present on surface 11a, with or without spacing between such arrays. That is, any given substrate may carry one, two, four or more arrays disposed on a front surface of the substrate and depending on the use of the array, any or all of the arrays may be the same or different from one another and each may contain multiple spots or features. For example embodiments may include 2n by 3n arrays on the carrier, where n is some integer such as 4, 8, or 16, or more generally 4x where x is an integer from about 1 to about 5, about 10, or about 20 or more (for example, about 5, about 6, about 7, about 8, about 9, about 10, about 11, about 12 or about 16).

The one or more arrays 12 usually cover only a portion of surface 11a, with regions of surface 11a adjacent the opposed sides 13c, 13d and leading end 13a and trailing end 13b of slide 10, not being covered by any array 12. A second or back surface 11a of slide 10, opposite first surface 11a, typically (though not necessarily) does not carry any arrays 12. Each array 12 may be designed for testing against any type of sample, whether a trial sample, reference sample, a combination of them, or a known mixture of biopolymers such as polynucleotides. Substrate 10 may be of any shape, as mentioned above.

As mentioned above, array 12 contains multiple spots or features 16 of biopolymers, e.g., in the form of polynucleotides. As mentioned above, all of the features 16 may be different, or some or all could be the same. The interfeature areas 17 could be of various sizes and configurations. Each feature carries a predetermined biopolymer such as a predetermined polynucleotide (which includes the possibility of mixtures of polynucleotides). It will be understood that there may be a linker molecule (not shown) of any known types between surface 11a and the first nucleotide.

Substrate 10 may carry on its surface an identification code, e.g., in the form of bar code (not shown in this embodiment) or the like printed on a substrate in the form of a paper label attached by adhesive or any convenient means. Identifiers such as optical, radiofrequency identification (“RF ID”) tags or magnetic identifiers could be used instead of bar codes. The identification code may contain information relating to array 12, where such information may include, but is not limited to, an identification of array 12, i.e., layout information relating to the array(s), etc.

Embodiments also include common carriers that include sets of chemical arrays such as described in copending, commonly assigned U.S. application Ser. No. 10/632,332, filed Jul. 31, 2003, and titled “Chemical Arrays On a Common Carrier”, which is incorporated herein by reference in its entirety. As such embodiments are described in the above noted U.S. application, they will not be described in great detail herein. In brief, such embodiments include a common carrier which may carry a set of arrays together. The set of chemical arrays may be separable into multiple sub-sets each with one or more arrays, where separation may take place before contact with a sample or after contact with a sample. The common carrier may include an indication of locations along which the carrier should be separated so as to separate the set of chemical arrays into multiple sub-sets each with one or more arrays. The common carrier may be rigid or flexible.

As will be described in greater detail below, the present invention may be employed in an easy and high throughput manner in the exposing of the arrays to one or more different or same fluids, such as one or more liquid samples, while the set of arrays is held together by the common carrier. For example, such may be accomplished automatically and all of the arrays of the common carrier may be contacted with the same or different fluid at the same time, without cross contamination of fluids between the arrays.

In certain embodiments, the common carrier may be a one-piece substrate having a surface on which the arrays are disposed. An indication of locations along which separating occurs may then include markings on the substrate prior to the separating. In certain other embodiments, the common carrier may include a substrate holder, and the sub-sets of arrays may each be carried on separate substrates mounted at different locations on the holder. In this situation the separating may simply be removing the separate substrates from the holder. The indication of the locations along which the carrier should be separated in this situation may be a visual indication of locations at which the separate substrates may be removed from the holder. For example, there may be a visible line between the separate substrates, or there may be some means for removal of each separate substrate which provides the visual indication. In any event, the apparatus may include multiple array identifiers which are positioned on the one-piece substrate or separate substrates before the separating such that after the separating each separated sub-set of arrays is carried on a separate substrate along with at least one of the array identifiers.

Various configurations of the set of arrays held together by the common carrier are possible. For example, the sub-sets of arrays which are separated may be arranged in two directions on the common carrier cross-wise to one another before the separating. The set of arrays on the common carrier before the separating may consist of 2n by 3n arrays on the carrier, where n is some integer such as 4, 8, or 16, or more generally 4x where x is an integer from about 1 to about 5, about 10, or about 20 or more (for example, about 2, about 3, about 4, about 5, about 6, about 7, about 8, about 9, about 10, about 11, about 12 or about 16). The common carrier may have a length and width which is equal to that of any common laboratory sample device, such as no greater than about 150 mm or about 130 mm, by about 100 mm or about 90 mm, to allow compatibility with the well known standard 96, 384, or 1536 well microtiter plate format.

As a result of the separating, the number of sub-sets may be any desired number as indicated by the visual indication of locations for the separating. For example, the set of arrays may be separated into 2, 4, 6, 8, 10, or 12 sub-sets on separate substrates which all may or may not have the same length and width (that is, the length is the same for all separate substrates, and the width is the same for all separate substrates, but the length and width for a given substrate may be the same or different). In one case the separated substrates may have a width and length such as 2.54 cm by 7.62 cm (1″ by 3″).

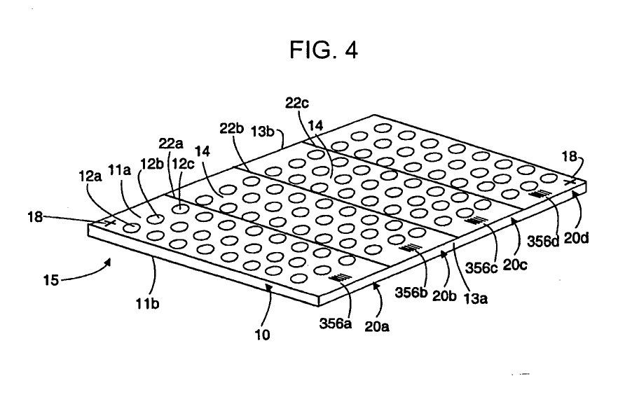

FIGS. 4-7 show embodiments of common carriers having sets of chemical arrays. FIGS. 4-7, show array assembly 15 which may be fabricated using any of those methods already described herein. Array assembly 15 includes a common carrier in the form of a one-piece substrate which can, for example, be in the form of a rigid substrate 10 (for example, a transparent non-porous material such as a single piece of glass or silica) carrying one or more arrays 12 (such as arrays 12a, 12b, 12c) disposed along a flat front surface 11a of substrate 10 and all separated by a same inter-array surface region 14 which surrounds each of arrays 12. Inter-array surface region can be considered to extend just beyond the outermost of arrays 12 in FIG. 4. The continuous region carrying arrays 12 includes the arrays 12 and inter-array region 14. Alternatively, substrate 10 may be flexible. Each array 12 occupies its own region on surface 11a which is co-extensive with the array (hence the regions do not extend into inter-array region 17). A back side 11b of substrate 10 typically (though not necessarily) does not carry any arrays 12.

In the configuration of FIG. 4 substrate 10 may have any suitable dimensions. For example, assembly 15 of FIG. 4 may have length and width dimensions of about 7.62 cm by about 10.16 cm and is shown carrying a set of ninety-six arrays 12 arranged in an eight by twelve array format in the same manner as wells of a standard ninety-six well microtiter plate. Front surface 11a of substrate 10 carries indications of locations along which substrate 10 should be separated in the form of straight line scores 22a, 22b, 22c. Scores 22 divide the set of arrays 12 into four sub-sets of twenty-four arrays (each of three by eight arrays) such that when substrate 10 is separated along scores 22, the resulting sections of separated substrate 20a, 20b, 20c, 20d (shown separated in FIG. 7) each have width and length dimensions of about 2.54 cm by about 7.62 cm (about 1″ by about 3″) and each carries one of the array sub-sets. Of course, more or less sub-sets may be used, each of any suitable dimension.

Substrate 10 further carries multiple (four in FIG. 4) array identifiers 356 in the form of bar codes on front surface 11a such that each section of separated substrate 20a-20d also carries an array identifier 356. Each identifier may be associated with each array 12 by being on the same substrate 10 and therefore having a fixed location in relation to identifier 356 from which relative location the identity of each array can be determined. Each array identifier can either carry array layout information or an identification linked to array layout information in a remote or non-remote memory, for each array on the section 20 which carries that identifier, as well as information on array features, all of which information can be used in a manner the same as described in U.S. Pat. No. 6,180,351 (which as mentioned above, is incorporated herein by reference). The substrate 10 may further have one or more fiducial marks 18 for alignment purposes, for example during array fabrication and reading and the like.

While ninety-six arrays 12 are shown in FIG. 4, it will be understood that substrate 10 may have any number of desired arrays 12 such as at least ten, thirty, fifty, one hundred, two hundred, or at least one thousand. For example there may be a total of 96, 384, or 1536 arrays laid out in an a 2n by 3n format where n is an integer from 4, 8, or 12 to 20, 30, or 50, such as n being 4, 8, or 16 (or some other whole multiple of 4). Scores 22 may still be positioned as in FIG. 4 to divide substrate 10 into four equal sections of separated substrate 20 when substrate 10 is separated along the scores 22. In this case each section of separated substrate 20 will have the same length and width as described above but each will then carry 2n by 3n/4 arrays 12 when n is a whole multiple of 4.

Depending upon intended use, any or all of arrays 12 may be the same or different from one another and each may contain multiple spots or features 16 of biopolymers in the form of polynucleotides, analogously to that described above. In the illustrated embodiment of FIG. 4, arrays 12 are generally round in shape (although other shapes, such as generally elliptical and square, are possible). As noted above, a typical array 12 may contain from more than five, ten, twenty, thirty, or one hundred features, or even at least one hundred, one thousand, two thousand, or at least four thousand features. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature of the same composition are excluded, the remaining features may account for at least 5%, 10%, or 20% of the total number of features).

Each array 12 may cover an area of less than 200 mm², 100 mm², or less than 50 mm², 20 mm², or less than 10 mm². Arrays 12 may be spaced apart from one another by a distance at least two, three, or four times the average distance between features within the arrays. In many embodiments, particularly when substrate 10 is rigid, it may be shaped generally as a rectangular solid as shown (although other shapes are possible). Other possible dimensions of substrate 10 include those in which it has a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. When substrate 10 is flexible, it may be of various lengths including at least 1 m, at least 2 m, or at least 5 m (or even at least 10 m). With arrays that are read by detecting fluorescence, the substrate 10 may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

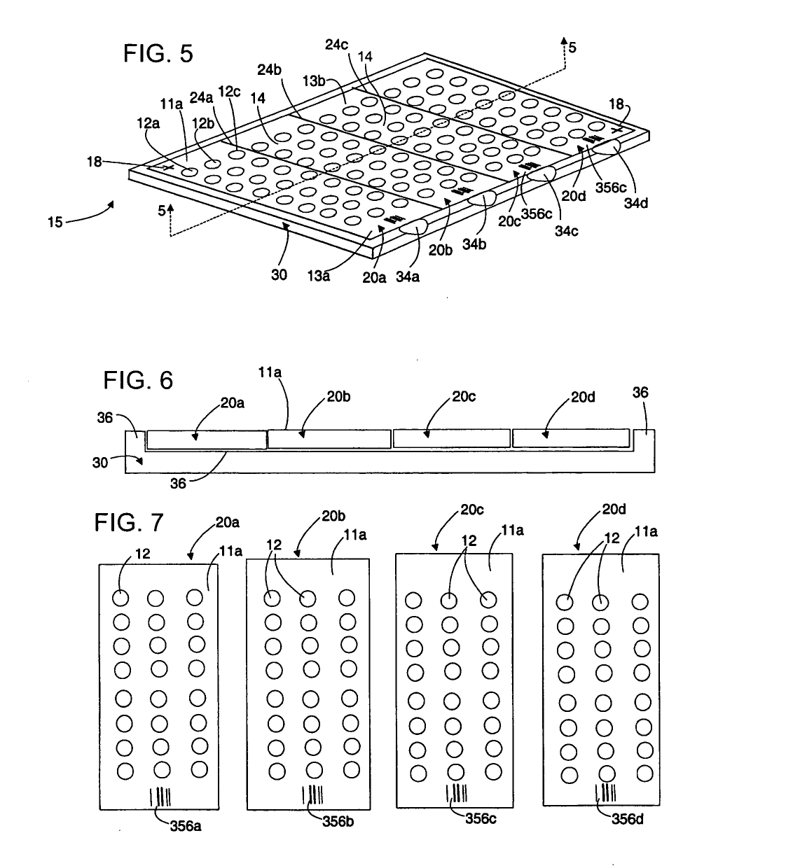

FIGS. 5 and 6 show another apparatus in the form of an alternate array assembly 15. In this embodiment the common carrier is in the form of a solid substrate holder 30 made of plastic, metal, or other suitable material. Substrate holder 30 has a recess in its upper surface into which previously separated sections 20a, 20b, 20c, 20d (as already described in connection with FIG. 4) can be snugly seated so as to be held by holder 30 in a position in which they are arranged in a series each abutting the next as shown in FIGS. 5 and 6 (though they could be arranged in a series each adjacent the next thereby permitting a slight spacing between them). A raised margin 36 surrounding this recess may be sized with as small as possible thickness (for example, less than about 20, about 10, about 5, or about 1 mm) so that the overall assembly 15 can have dimensions the same or similar to those already described above in connection with one-piece substrate 10 of FIG. 4. Indications of the locations along which separating should take place includes a visual indication of locations at which separate substrates may be removed, in the form of separation lines 24a, 24b, 24c which are visible as a result of sections 20a through 20d being already separated. Additionally, another visual indication of locations at which separate sections of substrate 20 may be removed from holder 30, may be provided by access points 34a, 34b, 34c, 34d which accommodate a user's fingertip to allow a section 20 to be pried upward and out of holder 30. The removed separated sections of substrate 20 again are shown in the top view of FIG. 7. Alternatively, in the embodiment of FIGS. 5 and 6 the sections 20a, 20b, 20c, 20d may not be previously separated in holder 30. That is, a rigid one-piece substrate such as that of FIG. 4 may instead be seated in holder 30. In this case scores 22 may serve as the visual indications of the locations along which separating should take place.

In any of the embodiments described above, the length and width of the carrier, whether it be the carrier of FIG. 1 or the common carrier (whether the one-piece glass substrate 10 or holder 30) of FIGS. 4-7 may be any suitable dimension. For example, in certain embodiments the length and width may be selected to be about 128 mm by about 85 mm which are about the same as those for the well known standard 96, 384, or 1536 well microtiter plate format.

Reservoir Plates

As noted above, the subject invention provides reservoir plates for use with chemical arrays. More specifically, the reservoir plates of the subject invention are employed in methods of contacting fluid with one or more chemical arrays and may be characterized as fluid contacting plates. The subject reservoir plates may be of particular use in the contacting of a fluid with one or more arrays of an array set. The reservoir plates provide significant advantages and benefits over fluid contacting techniques previously used, as will be described in greater detail below. One particular feature of the subject reservoir plates is that, when used, there is no cross-contamination of fluids between fluid contacted with one array, e.g., an array of a set of arrays, and fluid contacted with any other arrays, e.g., any other arrays of a set of chemical arrays. In other words, embodiments of the subject invention provides devices for contacting and maintaining a fluid at a defined area of an array substrate, e.g., about a particular chemical array of an array assembly.

The reservoir plates may be employed with array assemblies and used in a variety of array assays including hybridization assays. Specific hybridization assays of interest with which the subject reservoir plates may be employed include: gene discovery assays, differential gene expression analysis assays; nucleic acid sequencing assays, and the like. Patents describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference.

Other array assays of interest with which the subject reservoir plates may be employed include those where the arrays are arrays of polypeptide binding agents, e.g., protein arrays, where specific applications of interest include analyte detection/proteomics applications, including those described in U.S. Pat. Nos. 4,591,570; 5,171,695; 5,436,170; 5,486,452; 5,532,128; and 6,197,599; as well as published PCT application Nos. WO 99/39210; WO 00/04832; WO 00/04389; WO 00/04390; WO 00/54046; WO 00/63701; WO 01/14425; and WO 01/40803; the disclosures of the United States priority documents of which are herein incorporated by reference.

The present inventors recognize that there exists a need to facilitate fluid handling with respect to chemical arrays and especially for arrays such as those described above (common carriers with sets of arrays, e.g., in the form of standard microtiter plates and the like) which provide a large number of arrays per substrate.

The present invention provides in one aspect reservoir plates for contacting fluid with one or more chemical arrays disposed on a surface of an array assembly, such as for contacting fluid to a set of chemical arrays held together by a common carrier, e.g., in the form of a standard microtitre plate or the like.

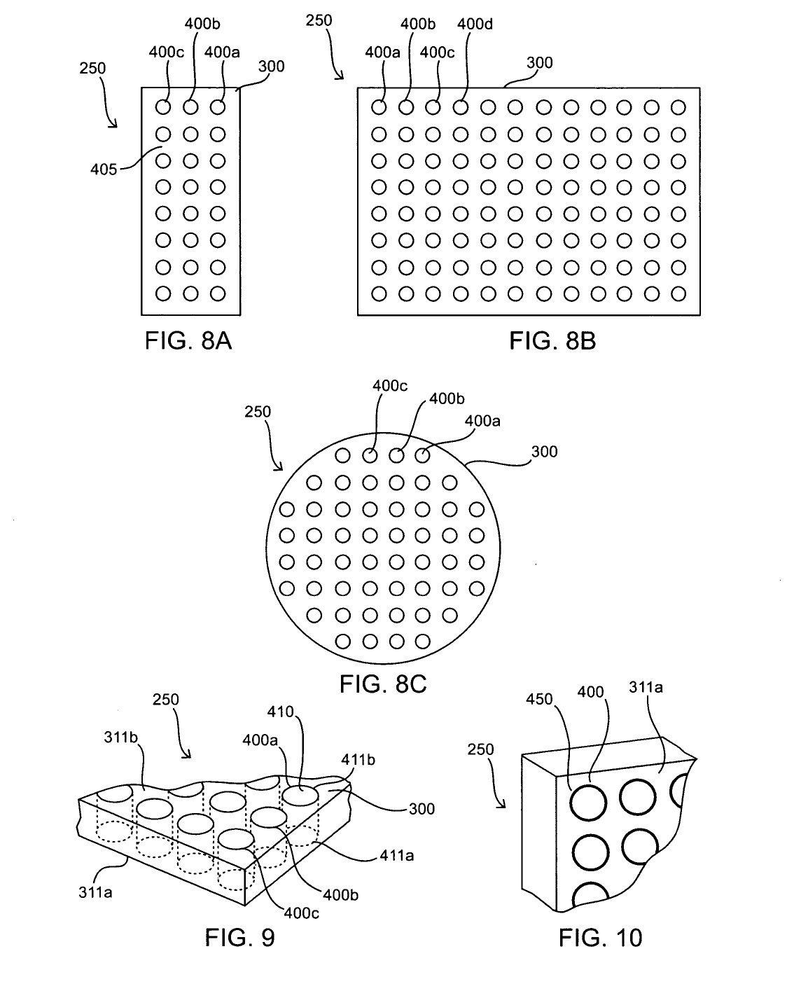

In the broadest sense a subject reservoir plates may be described as a support (e.g., planar support) that includes one or more holes or bores through the support. The holes may be configured to align with a chemical array when the array and the reservoir plate are operatively positioned relative to each other to provide an array assembly/reservoir plate fluid handling structure. The subject reservoir plates can accommodate a wide range of array formats, e.g., by configuring a given reservoir plate to correspond to a given array assembly configuration and/or by only utilizing certain holes of the reservoir plate to accommodate a particular array configuration. FIGS. 8A, 8B, 8C show top views of exemplary embodiments of reservoir plates 250 that include support 300 having one or more holes 400 that extend through the entire thickness of the support and are open at both ends so there is an opening from one side of the support to an opposite side of the support via a hole that traverses through the support. FIG. 9 shows a portion of a plate and FIG. 10 shows a surface 311a of the reservoir plate portion shown in FIG. 9.

As noted above, reservoir plates according to the subject invention include a support. As shown in the figures, support 300 includes a first side or array facing side 311a and a second or non-array facing side 311b that is opposite side 311a. The support may assume a variety of shapes and sizes, where a given support may be configured (e.g., sized, shaped, etc.) to be operatively positioned relative to an array assembly so that a fluid may be contacted with a defined region of the array assembly by introducing the fluid through one of the holes of the reservoir plate. For example, embodiments include reservoir plates that are configured (e.g., sized and shaped) such that when a given reservoir plate is operatively positioned relative to an array assembly, at least one of the holes of the plate is aligned with (positioned over) at least one array of the array assembly. In many embodiments, reservoir plates may be configured (e.g., sized and shaped) such that when a given reservoir plate is operatively positioned relative to an array assembly, all of the holes of the plate are aligned with (positioned over) substantially all, including all, of the arrays of the array assembly such that one hole of the reservoir plate overlies one array of the array assembly. In this aspect, the same or different fluid may be precisely contacted with one or more arrays of an array assembly through one or more holes of a reservoir plate in a high throughput manner.

One or more surfaces (e.g., the array-facing surface) of a reservoir plate may include certain structural features, e.g., may include recessed structures, elevated structures, channels, orifices, guides, hydrophobic areas, hydrophilic areas, fluid retaining barriers such as gaskets and the like, etc. For example, a fluid retaining barrier may encircle an array-facing opening of a hole. In certain embodiments, the side wall of the holes of a reservoir plate may incorporate a surface treatment. For example the side walls of the holes may be hydrophilic or may be rendered hydrophilic or may include other surface treatments or structures.

The particular shape of a support is typically, though not always, dictated at least in part by the configuration of an array assembly with which it may be used such that the shape of a given plate may be the same or similar to the shape of an array assembly with which it is designed to be used and as such plates may have shapes analogous to shapes described above for array assemblies. In any event, the shapes of the supports may range from simple to complex. In certain embodiments, the supports may be square, rectangular, oblong, oval or circular shape, elliptical, etc., as well as other geometric shapes and irregular or complex shapes.

Likewise, the size of the supports may vary depending on a variety of factors, including, but not limited to, the particular array assembly to which it is designed to be used, and as such supports may have sizes the same as or similar to sizes described above for array assemblies. In certain embodiments, supports may have width and length dimensions equal to that of any common laboratory sample device, such as no greater than about 150 mm or about 130 mm, by about 100 mm or about 90 mm, such as analogous to dimensions of well known standard 96, 384, or 1536 well microtiter plates. Embodiments include supports having width and length dimensions of about 2.54 cm by about 7.62 cm (about 1″ by about 3″) in certain embodiments. In certain embodiments, e.g., for use with arrays configured in the form of standard microtitre plates (e.g., having 96 arrays), a support may have dimensions of about 120 mm×about 100 mm, e.g., about 127.7 mm×about 85.35 mm. Such embodiments thus support, for example, a 1″×3″ array substrate carrier, the four de-mountable 1″ by 3″ substrates held together in a common carrier (array holder of FIG. 5), as well as the one-piece substrate carrier of FIG. 4 (e.g., having dimensions of about 3″ by about 4″). The thickness of the supports may range from about 0.5 mm to about 1 cm, e.g., from about 0.5 mm to about 10 mm, e.g., from about 0.5 mm to about 5 mm.

The plates may be made from any suitable material and are usually chosen with respect to the conditions to which the supports may be exposed, e.g., the conditions of any treatment or handling or processing that may be encountered in the use of the supports, e.g., hybridization assays, protein binding assays, washings, etc. One or more materials may be used to fabricate the supports such that a plurality of materials may be employed. Examples of materials which may be used to fabricate the subject supports include, but are not limited to, metals such as stainless steel, aluminum, and alloys thereof; polymers, e.g., plastics and other polymeric materials such as poly (vinylidene fluoride), poly(ethyleneterephthalate), polyurethane, e.g., nonporous polyurethane, fluoropolymers such as polytetrafluoroethylene (e.g., Teflon®), polypropylene, polystyrene, polycarbonate, PVC, and blends thereof; siliceous materials, e.g., glasses, fused silica, ceramics and the like. The supports may be flexible or rigid.

The supports may be fabricated from a “composite,” i.e., a composition made up of different or unlike materials. The composite may be a block composite, e.g., an A-B-A block composite, an A-B-C block composite, or the like. The composite may be a heterogeneous combination of materials, i.e., in which the materials are distinct from separate phases, or a homogeneous combination of unlike materials. As used herein, the term “composite” is used to include a “laminate” composite. A “laminate” refers to a composite material formed from several bonded layers of identical or different materials.

As described above, the supports include at least one hole 400 and oftentimes a plurality of holes (400a, 400b, 400c . . . ), e.g., arranged in an array pattern as will be described below and separated by a hole spacing 405. Each hole of a support extends in a thickness dimension of the support and each hole is open at both ends, i.e., the holes are through holes or bores through a support, i.e., open channels or passages that extend through the support. Each hole 400 has a side wall 410 adjacent to a first or array-facing open end 411a and at an opposite end adjacent to a second open end 411b. The first and second open ends, adjacent respective surfaces 311a and 311b of support 300, provide access to an operatively positioned array assembly. Specifically, second open end 411b configured for receiving a volume of fluid to be contacted with an array to which the hole is to be operatively associated, which fluid is thus contacted with an array via open end 411a.

The number of holes may vary and may depend on the particular application with which the reservoir plate is used, the particular array assembly with which it is used etc. The number of holes may range from about 1 to about 500 or more, e.g., 1 to about 100. In many embodiments, the number of holes roughly corresponds to, i.e., is the same as or similar to, the number of arrays of an array assembly with which it is designed to be used. As such, if the array assembly includes 1 chemical array, the reservoir plate may include 1 hole, if the array assembly includes 8 chemical arrays, the reservoir plate may include 8 holes, if the array assembly includes 10 chemical arrays, the reservoir plate may include 10 holes, if the array assembly includes 16 chemical arrays, the reservoir plate may include 16 holes, if the array assembly includes 96 chemical arrays, the reservoir plate may include 96 holes, etc. For example, plates may include about 2n by about 3n holes, where n is some integer such as about 4, about 8, or about 16, or more generally 4x where x is an integer from about 1 to about 5, about 10, or about 20 or more (for example, about 2, about 3, about 4, about 5, about 6, about 7, about 8, about 9, about 10, about 11, about 12 or about 16). The number of holes need not match exactly to the number of arrays of an array assembly with which it is to be used, and may be more or less. For example, it may not be desirable to contact all of the arrays with fluid at the same time, or the like.

The holes may be arranged in any suitable configuration and may be based at least in part on the particular array assembly with which it is designed to be used etc. For example, holes may be present as a pattern, where the pattern may be in the form of organized rows and columns of holes, e.g. a grid of holes, across the support, a series of curvilinear rows across the support, and the like. As noted above, embodiments include reservoir plates having holes in an array pattern. A reservoir plate may be designed to be used with an array assembly having a grid pattern of arrays such as shown in FIGS. 4, 5 and 6, and thus the reservoir plate may have holes in the same or analogous grid pattern. For example, a reservoir plate may be designed to be used with an array assembly having 96 arrays arranged in a grid pattern (e.g., FIGS. 4 and 5), and thus the reservoir plate may include about 96 holes arranged in the same or analogous grid pattern as the 96 arrays with which it is intended to be used. Likewise, a reservoir plate may be designed to be used with an array assembly having less than 96 arrays, such as for example for use with a sub-set of a set of arrays such as the 24 arrays of any one of separated substrate 20a, 20b, 20c, 20d (FIG. 7), and thus the plate may include about 24 holes arranged in the same or analogous grid pattern as the 24 arrays with which it is intended to be used. In certain embodiments, a reservoir plate may be used with an array assembly having 8 arrays or 16 arrays (e.g., arranged in two parallel columns of 4 or 8 arrays, respectively, and thus the reservoir plate may include about 8 holes or 16 holes, respectively, arranged in the same or analogous pattern as the 8 or 16 arrays with which it is intended to be used.

While the holes are shown as circular in the figures herein, the holes are not limited to any particular shape and may be square, rectangle, oval, etc. In many embodiments, the holes are the same as or similar to the shape of the arrays with which the reservoir plate is designed to be used.

The diameter or width of the holes may be any suitable width any may be constant throughout the hole or may change, e.g., the diameter may increase or decrease from opening 411b to 411a. In many embodiments, a hole will be limited in size to be the same as or similar to the size of a chemical array it is to overlie. In other words, while in certain embodiments holes may be large enough such that, when a reservoir plate is operatively positioned relative to an array assembly, the holes are sized so that a single hole is positioned over more than one array, in many embodiments when a reservoir plate is operatively positioned relative to an array assembly the holes are sized so that a single hole is positioned over just one array.

In certain embodiments, the perimeter of an opening of one or more holes of a reservoir plate may be surrounded by a fluid retaining structure 450 such as a gasket or surface treatment (hydrophilic) or the like which may serve to help maintain a fluid at a particular location of an array assembly, e.g., about a particular array, such that the fluid retaining structure may be contacted with the array assembly to provide a wall about the array. Such is shown in FIG. 10 which shows a partial view of surface 311a of reservoir plate 250.

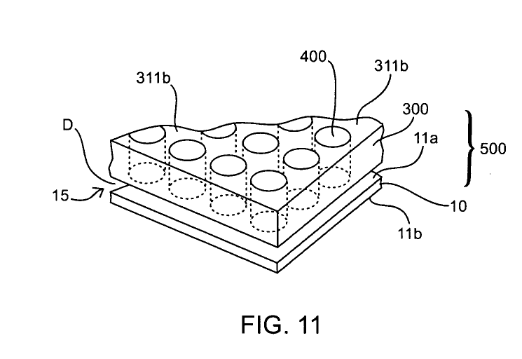

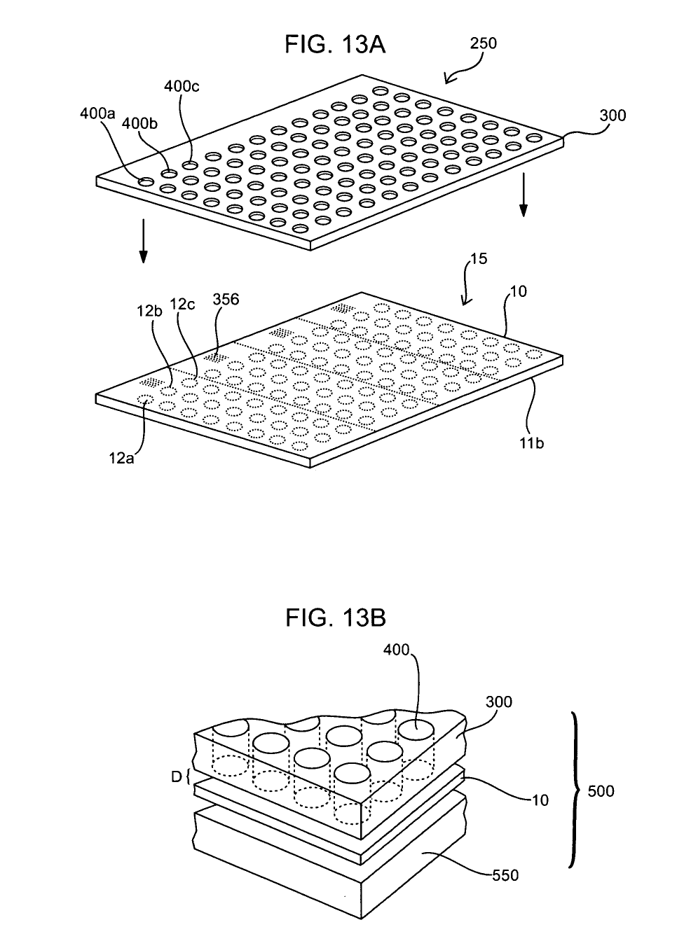

In use, a reservoir plate is positioned in opposition and relative to an array assembly as shown in FIG. 11. As will be described in greater detail below, a reservoir plate may be directly contacted with a surface of the substrate of the array assembly or may be spaced-apart from the array substrate a distance such as a capillary distance. Spacing the reservoir plate a distance from the array assembly. In certain embodiments, physically touching the reservoir plate to the array assembly may damage the arrays (and/or the array substrate upon which the arrays are disposed), compromising array assay results. Accordingly, spacing the reservoir plate a distance from the array assembly preserves the integrity of the arrays (as well as the array substrate which may be fragile). This embodiments of the subject invention also provides chemical array fluid contacting structures 500 that include a reservoir plate operatively positioned relative to an array assembly. The two components of the structure may be maintained in operative relation with respect to one another manually, or with the aid of a housing, fasteners, clamps, spacers, friction fit, snap fit, and the like. In many embodiments, each hole of the reservoir plate is aligned over at least one, and in many embodiments only one, array of the array assembly of the structure. When so positioned, the reservoir plate enables a volume of fluid to be contacted to and maintained about an array of the array assembly (see for example FIG. 13C). The volume of fluid may differ depending on the particular size of the holes of the reservoir plate, distance (if any) between a surface of the reservoir plate and an opposing surface of the array assembly. In certain embodiments, the volume of fluid V may range from about 5 microliters to about 500 microliters, e.g., from about 10 microliters to about 300 microliters.

Methods of Contacting a Fluid with an Array Assembly

Methods for contacting a fluid with a defined region of a surface of an array assembly are also provided. In many embodiments, discrete amounts of fluid may be introduced to a plurality of discretely defined regions (e.g., regions separated by inter-array spacings) of the array assembly at the same time, where the fluids contacted with different regions may be the same or may be different, and in certain embodiments may be different amounts. Defined regions to which fluid is contacted include one or more chemical arrays, where in many embodiments fluid is contacted with a plurality of chemical arrays at the same time without cross contamination between the fluids contacted with the different arrays.

The subject invention may be employed in a variety of array assays including hybridization assays. Specific hybridization assays of interest which may be practiced using the subject invention include: gene discovery assays, differential gene expression analysis assays; nucleic acid sequencing assays, and the like. Patents describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference.

Other array assays of interest include those where the arrays are arrays of polypeptide binding agents, e.g., protein arrays, where specific applications of interest include analyte detection/proteomics applications, including those described in U.S. Pat. Nos. 4,591,570; 5,171,695; 5,436,170; 5,486,452; 5,532,128; and 6,197,599; as well as published PCT application Nos. WO 99/39210; WO 00/04832; WO 00/04389; WO 00/04390; WO 00/54046; WO 00/63701; WO 01/14425; and WO 01/40803; the disclosures of the United States priority documents of which are herein incorporated by reference.

In general, the subject methods include positioning a subject reservoir plate relative to a surface of an array assembly and introducing fluid through at least one hole of the reservoir plate such that the fluid contacts a defined region of the array assembly. Embodiments include reservoir plates that are operatively positioned relative to a surface of an array assembly such that a capillary space is provided between the reservoir plate and the array assembly surface. Embodiments also include reservoir plates that are operatively positioned relative to a surface of an array assembly such that the reservoir plate or a portion thereon is directly contacted with a surface of the array assembly.

In certain embodiments, a fluid may be contacted with one or more arrays of a chemical array set not having been previously exposed to a sample. For example, a contacted fluid may include the sample. Embodiments also include contacting a fluid with one or more chemical arrays of a set having been previously exposed to a sample. For example, a fluid may be a wash fluid. Certain embodiments include, before or after contact with a fluid, separating a fluid-contacted set of chemical arrays into multiple sub-sets each with one or more arrays.

Different embodiments of the present invention may provide any one or more of the following, or other, useful benefits. For example, embodiments of the subject invention provide high throughput fluid contacting methods for use with contacting arrays arranged in a particular high throughput format (such as a set of separable chemical arrays). In this manner, the arrays may be provided in a high throughput manner and processed using the high throughput fluid contacting methods of the present inventions, and then the arrays may be separated to provide arrays in a different format (e.g., subsets of arrays) so that the arrays may be read in an apparatus which may not accommodate the particular high throughput format, but which may accommodate the format of the sub-set of arrays.

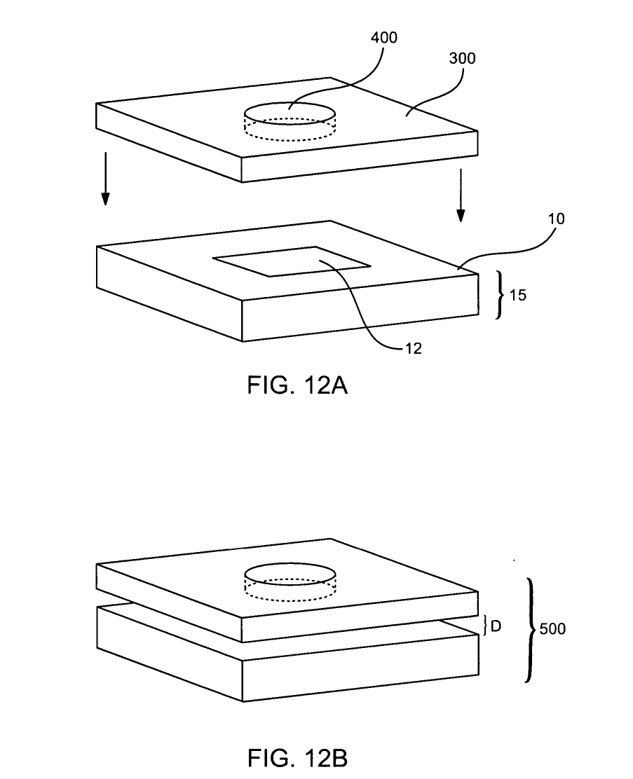

A first step in the subject methods includes positioning an array assembly and a reservoir plate in operative relation to one another. FIGS. 12A and 12B show one embodiment wherein the reservoir plate includes a single hole 400 through support 300 such that the reservoir plate is configured to be positioned in opposition to array 12, such as array 12 of the array assembly of FIG. 1. FIG. 12B shows the reservoir plate in operative position relative to the array assembly to provide fluid contacting structure 500. In this embodiment, the two are spaced-apart a distance “D” from each other to provide a gap between the support and the array assembly (i.e., the array assembly and the support are not in physical contact with each other), however in certain other embodiments the two may be in physical contact with each other.

As shown in the figure, distance D is the distance between the two opposing surfaces of a reservoir plate and an array assembly. This spacing may be maintained by any convenient method, e.g., spacers (see for example spacers 470 of FIG. 13C) may be positioned between the reservoir plate and the array assembly, or the two may be maintained a distance from each other by an automated handling system which positions and maintains the two components a distance from each other, as will be described in greater detail below. In many embodiments, the distance between the reservoir plate and a surface of the array assembly is a capillary distance. By capillary distance is meant a gap small enough to hold capillary. In this manner, the capillary distance enables fluid to be maintained between the reservoir plate and a surface of the array assembly at a defined region by capillary forces, where the capillary distance is any suitable distance that provides a capillary. In certain embodiments, the capillary gap may be less than about 450 μm, e.g., less than about 350 μm, e.g., less than about 300 μm, e.g., less than about 250 μm. In this manner, fluid may be retained at defined regions of the array assembly (about the arrays (see for example FIG. 13C) due at least in part to the capillary forces provided by the capillary distance of the spaced-part reservoir plate. Furthermore, due to the attraction of fluid (such as hybridization buffer or the like) to the array features and surrounding areas due to feature and surrounding area hydrohilicity (and to the side walls of the holes if the side walls are hydrophilic), fluid is contained at defined areas about the array features. This fluid containment is further augmented by the air about the arrays provided by the capillary gaps, which air is hydrophobic.

Another embodiments is shown in FIGS. 13A and 13B. In this embodiment, array assembly 15 includes 96 chemical arrays, such as array assembly 15 of FIG. 4 (array assembly of FIG. 5 may also be contemplated in this regard). As shown, reservoir plate 250 includes support 300 having 96 holes 400a, 400b, 400c . . . arranged in a pattern analogous to the pattern of 96 arrays. Reservoir plate 250 is thus configured to be positioned in opposition to a surface 11a of array assembly 15 having a plurality of arrays 12a, 12b, 12c . . . such that each hole of reservoir plate 250 overlies each and only one array of array assembly 15. FIG. 13B shows a partial view of the reservoir plate and array assembly of FIG. 13A wherein the reservoir plate is in position relative to the array assembly and a capillary distance is provided therebetween which is maintained by appropriately sized (e.g., less than about 250 μm or less) spacers 470 (see FIG. 13C) to provide a capillary distance. FIG. 13B also shows optional heater element 550 which may be employed, e.g., to promote incubation of the fluid, e.g., a fluid sample) with the arrays.

Accordingly, a surface of a reservoir plate is positioned in operative relation to a surface of an array assembly where in many embodiments a gap is maintained between the two that is small enough to hold capillary. Once operatively positioned, a fluid may be contacted to one or more arrays of the array assembly by introducing the fluid through one or more holes (depending on the number of arrays intended to be contacted with fluid) of the reservoir plate. Such will now be described with reference to FIG. 13C which shows a cross-sectional view through fluid contact structure 500 of FIG. 13B. Such description is for exemplary purposes only and is in no way intended to limit the scope of the invention as it will be apparent that the described methods are not limited to the embodiment of FIGS. 13A-13C and may be employed with any embodiments.

A volume V of fluid F (represented as stippling) is introduced into each hole using any suitable fluid transfer element such as a pipettor, needle, and the like, either manually or automatically (e.g., using a robotic pipetting system or the like). Any one or more, including all, of the holes of the reservoir plate may be used at any one time such that in certain embodiments fluid may be introduced to some or all of the holes of a reservoir plate at the same time. The subject methods are not limited to any particular fluid and as such any suitable fluid may be employed in the practice of the subject invention. Typically, the fluids are fluids employed in at least some respect in an array assay such as a hybridization assay, protein binding assay, and the like. Fluids include buffers such as hybridization buffers and the like, fluidic samples, wash fluids, and the like.

FIG. 13C shows fluid transfer elements 560, such as which may be part of a robotic pipettor, which fills each hole with fluid such as, e.g. sample-containing hybridization buffer. Once introduced into the holes of the reservoir plate, the fluid is maintained at each respective chemical array by capillary forces. Optional hydrophobic rings/regions around each array and/or fluid retaining structures associated with each hole may also be employed to ensure fluid is maintained at respective arrays and cross-contamination does not occur between fluids about different arrays does not occur, as well as the inherent hydrophilicity of the array features and surrounding areas and the inherent hydrophobicity of the air “A” that surrounds the arrays due to the spacing of the reservoir plate a capillary distance from the surface of the array assembly. While some mixing may be used, typically no gross motion mixing is required. Optional heating element may be 550 employed to incubate sample with the arrays, in those instance where the fluid includes a sample of target molecules as described above. Of course, the fluid may be any fluid such as wash fluid or the like, as previously mentioned. In certain embodiments, once fluid is contacted and maintained about an array, holes may be covered to prevent evaporation. For example, a plastic film, foil or other suitable seal may be provided about the holes to reduce evaporation from the top (non-array facing end 411b) of the holes.

As noted above, embodiments include contacting sample with an array. In these assays, a sample to be contacted with an array may first be prepared, where preparation may include labeling of the targets with a detectable label, e.g. a member of signal producing system. Generally, such detectable labels include, but are not limited to, radioactive isotopes, fluorescers, chemiluminescers, enzymes, enzyme substrates, enzyme cofactors, enzyme inhibitors, dyes, metal ions, metal sols, ligands (e.g., biotin or haptens) and the like. Thus, at some time prior to the detection step, described below, any target analyte present in the initial sample contacted with the array may be labeled with a detectable label. Labeling can occur either prior to or following contact with the array. In other words, the analyte, e.g., nucleic acids, present in the fluid sample contacted with the array may be labeled prior to or after contact, e.g., hybridization, with the array. In some embodiments of the subject methods, the sample analytes e.g., nucleic acids, are directly labeled with a detectable label, wherein the label may be covalently or non-covalently attached to the nucleic acids of the sample. For example, in the case of nucleic acids, the nucleic acids, including the target nucleotide sequence, may be labeled with biotin, exposed to hybridization conditions, wherein the labeled target nucleotide sequence binds to an avidin-label or an avidin-generating species. In an alternative embodiment, the target analyte such as the target nucleotide sequence is indirectly labeled with a detectable label, wherein the label may be covalently or non-covalently attached to the target nucleotide sequence. For example, the label may be non-covalently attached to a linker group, which in turn is (i) covalently attached to the target nucleotide sequence, or (ii) comprises a sequence which is complementary to the target nucleotide sequence. In another example, the probes may be extended, after hybridization, using chain-extension technology or sandwich-assay technology to generate a detectable signal (see, e.g., U.S. Pat. No. 5,200,314).

In certain embodiments, the label is a fluorescent compound, i.e., capable of emitting radiation (visible or invisible) upon stimulation by radiation of a wavelength different from that of the emitted radiation, or through other manners of excitation, e.g. chemical or non-radiative energy transfer. The label may be a fluorescent dye. Usually, a target with a fluorescent label includes a fluorescent group covalently attached to a nucleic acid molecule capable of binding specifically to the complementary probe nucleotide sequence.

Following sample preparation (labeling, pre-amplification, etc.), the sample may be introduced to the array using a subject reservoir plate as described. The sample is contacted with the array under appropriate conditions to form binding complexes on the surface of the substrate by the interaction of the surface-bound probe molecule and the complementary target molecule in the sample. The presence of target/probe complexes, e.g., hybridized complexes, may then be detected. In the case of hybridization assays, the sample is typically contacted with an array under stringent hybridization conditions, whereby complexes are formed between target nucleic acids that agent are complementary to probe sequences attached to the array surface, i.e., duplex nucleic acids are formed on the surface of the substrate by the interaction of the probe nucleic acid and its complement target nucleic acid present in the sample. A “stringent hybridization” and “stringent hybridization wash conditions” in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO4, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions that set forth the conditions which determine whether a nucleic acid is specifically hybridized to a surface bound nucleic acid. Wash conditions used to identify nucleic acids may include, e.g.: a salt concentration of about 0.02 molar at pH 7 and a temperature of at least about 50° or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

A specific example of stringent assay conditions is hybridization at 65° C. in a salt based hybridization buffer with a total monovalent cation concentration of 1.5 M (e.g., analogous to that described in U.S. patent application Ser. No. 09/655,482 filed on Sep. 5, 2000, the disclosure of which is herein incorporated by reference) followed by washes of 0.5×SSC and 0.1×SSC at room temperature.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

The array is incubated with the sample under appropriate array assay conditions, e.g., hybridization conditions, as mentioned above, where conditions may vary depending on the particular biopolymeric array and binding pair.

Once the incubation step is complete, the array is typically washed at least one time as noted above to remove any unbound and non-specifically bound sample from the substrate, generally at least two wash cycles are used. Washing agents used in array assays are known in the art and, of course, may vary depending on the particular binding pair used in the particular assay. For example, in those embodiments employing nucleic acid hybridization, washing agents of interest include, but are not limited to, salt solutions such as sodium, sodium phosphate (SSP) and sodium, sodium chloride (SSC) and the like as is known in the art, at different concentrations and which may include some surfactant as well.

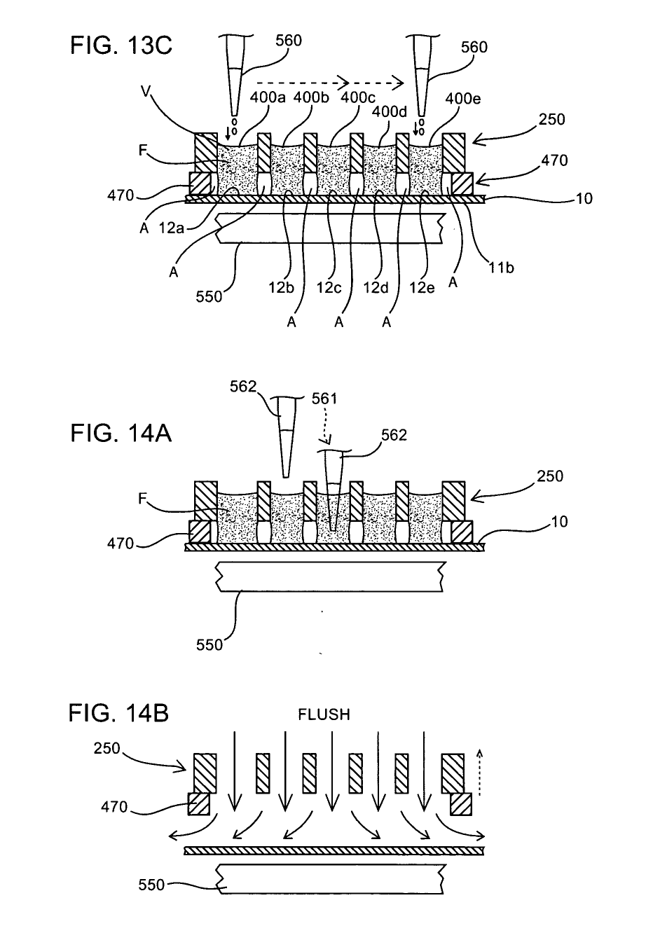

Once sample incubation is complete, removal of the sample and optional additional fluid contact, e.g., for washing of the arrays of the array assembly, may be performed as now described with respect to FIGS. 14A and 14B. In those embodiments wherein arrays are associated with a common carrier such as a set of arrays, the set may remain associated with the common carrier during washing of the arrays or may be dis-associated. For example, in those embodiments that include a set of chemical arrays held together by a common carrier in the form of a one-piece substrate, the set may be separated into multiple sub-sets of array prior to washing or may be kept together as a set and optionally separated sometime after washing. In those embodiments that include a set of chemical arrays held together by a common carrier in the form of an array holder, the arrays may or may not be separated from the common carrier prior to washing.

After a fluid has been suitably incubated with the one or more arrays, the fluid is removed. This may be accomplished by using one or more fluid transfer elements which may be the same or different from fluid transfer elements 560 used to deposit the fluid. FIG. 14A shows fluid transfer element 562 going from a first position to a second fluid removal position according to arrow 561. Fluid may be removed from each respective array at the same or different time. Fluid removal may be manual or automatic (e.g., using an automated robotic pipettor system).

Once fluid is removed from each array, the reservoir plate may be separated from the array assembly as shown in FIG. 14B and the array surface of the array assembly may be contacted with wash fluid via the reservoir plate to wash-off any non-specifically bound target material and any first fluid still left, where suitable wash fluids and wash conditions are described above. In certain embodiments as shown in FIG. 14B, the array surface is flushed with a volume of wash fluid by contacting the arrays with wash fluid introduced through one or more holes of the reservoir plate. The volume of wash may be a high volume such as about 100× to about 200× the volume of the hybridization buffer. For example, if about 50 microliters of sample-containing hybridization buffer may be retained about an array using by a hole of a reservoir plate, washing of the array may include about 100× as much fluid (e.g., about 5000 microliters to wash). During washing, the distance between the reservoir plate and the array surface of the array assembly may be increased, e.g., to a few millimeters or more. In certain embodiments, prior to contacting the arrays with wash fluid, the array assembly may be rotated to vertical with respect to ground to assist in the removal of any of the first-contacted fluid (e.g., sample) that may remain about the arrays.

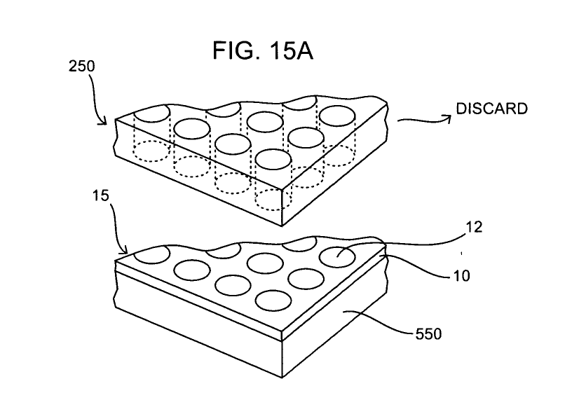

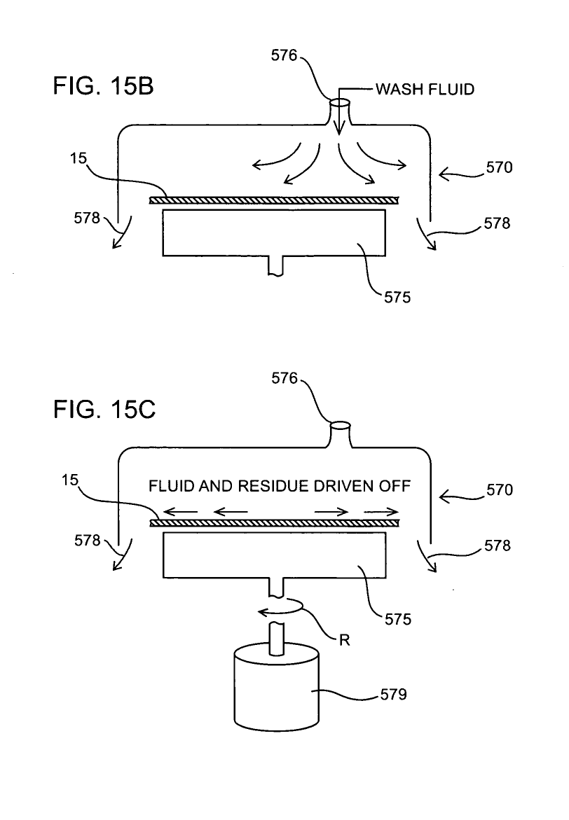

An alternative wash method is now described with reference to FIGS. 15A-15C. After any excess of the previously deposited fluid is removed, e.g., through the holes of the reservoir plate, e.g., by an automated robotic pipettor or the like, the reservoir plate may be removed all-together from the array assembly and re-used for another application or discarded. Accordingly, embodiments of the subject invention contemplate re-usable reservoir plates (e.g., made of re-useable material such as a suitable metal or metal alloy and the like) and one-time or consumable reservoir plates (e.g., made of consumable plastic or the like). Once the reservoir plate is suitably separated from the array assembly as shown in FIG. 15A, the array assembly may be substantially surrounded or placed inside of a flush/wash hood 570 as shown in FIG. 15B. Chamber 570 is configured to deliver wash fluid to the array for washing by introducing a wash fluid (or the like) through a wash fluid inlet 576 of the hood and dispose of the fluid through one or more wash fluid outlets 578, which may be in communication with a drain, discard reservoir, or the like. The array assembly is positioned in the hood on a suitable base 575 which in certain embodiments is an optionally rotatable base, e.g., rotatable about 360° about a vertical axis of the base. Once the array assembly is flushed with wash fluid the array assembly one or more times, the array assembly may be spun dry by rotating the base upon which it is positioned as shown in FIG. 15C to remove any wash fluid that may remain on the array assembly and dry the array assembly. As shown in FIG. 15C, base 575 is operatively associated with a drive motor 579 for rotating the array assembly in a suitable manner in the direction R to dry the array assembly. The drive motor may be a 1800 RPM drive motor capable of providing G-forces on the array assembly in the range of about 100 to about 300, e.g., about 200 Gs. Drying may also be accomplished exposing the array assembly to a nitrogen or other inter gas to dry the arrays. The array assembly, e.g., a set of arrays, while still on the common carrier, could be stored in ambient atmosphere or under controlled conditions (for example, for at least 10 minutes, 30 minutes, 1 hour, 5 hours, or at least 24 hours) in a chamber having an inert gas or other atmosphere free of contaminants and which blocks out at least 25% or 50% of total light between 500 to 200 nm) until shortly before reading.

Once dry, the arrays may be read. If the array assembly includes arrays held together by a common carrier, at this point sections 20 may be separated such as by breaking the substrate 10 along scores 22 in the case of array assembly 15 of FIG. 4, or in the case of array assembly 15 of FIG. 5 simply by inserting a fingertip at access points 34 and prying each section 20 upward out of holder 30. In either event separated sections 20 as shown in FIG. 7 are obtained. In a variation, not all separated section need be the same size or carry the same number of arrays. For example, in the array assembly of FIG. 4 or FIG. 5 only one section 20 may be separated from the remainder (that is, the three other sections may be attached together in the embodiment of FIG. 4 or seated in holder 30 in the embodiment of FIG. 5). This may be particularly convenient in the event that each of the unseparated sections have not yet been exposed to a sample. In this manner, a customer may just use the arrays on a separable section one or more at a time by exposing that section or sections to one or more samples, then separating and reading them, then repeating this process one or more times for previously unseparated sections. However, in many situations all of the arrays on all sections 20 may all have been exposed to one or more samples prior to any separation of a section 20. In certain embodiments, a set of arrays may not be separated and may be read un-separated. A reader may be used that is configured to read an entire set of chemical arrays, e.g., array assembly 15 of FIG. 4, or array assembly 15 of FIG. 5 without separating the set.

Note that leaving the sections 20 together on the common carrier up until just prior to reading them, can be used in a manner which provides one or more advantages. For example, a user could safely assume that all sections 20 on a common carrier are from the same fabrication run and that they all encountered the same environmental conditions prior to reading (for example, during sample exposure for hybridization, and during wash and storage both before and after sample exposure). Thus, a user could safely assume that sections 20 were not stored or processed separately since they were physically connected by being together on the common carrier, from the time an array assembly was received by the user (or even from the time the sections were fabricated and shipped from a fabrication location to the user) up to the point of their separation immediately preceding their reading.

Regardless, the arrays, such as separated sections 20, may then be prepared for reading. Depending upon the reader, if sections 20 are provided, sections 20 may be read in the format as shown in FIG. 7 or inserted into a suitable holder which may be required by the reader apparatus. Reading of may be accomplished by illuminating an array and reading the location and intensity of resulting fluorescence at each feature of the array to obtain a result. In addition to reading arrays such as the arrays 12 on a section 20, a reader may also read the identifier 356 (if present) on the same substrate section 20. The identifier may be used to retrieve the array layout for each array 12 on the same section 20 carrying that identifier 356, from a local or remote memory in a manner such as described in U.S. Pat. No. 6,180,351. One particular reader is disclosed in U.S. Pat. No. 6,406,849. Further details of such readers are disclosed in U.S. Pat. No. 6,320,196 and U.S. Pat. No. 6,486,457, the disclosures of which are herein incorporated by reference. Another particular reader that may be used is the AGILENT MICROARRAY SCANNER manufactured by Agilent Technologies, Palo Alto, Calif. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. Nos. 6,251,685; 6,221,583, the disclosure of which is herein incorporated by reference, and elsewhere).

Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample or whether or not a pattern indicates a particular condition of an organism from which the sample came). For example, the read raw signal data which is read from the arrays may be processed such as by feature extraction and further processing as desired. Examples of feature extraction programs for which instructions or parameters may be provided, include methods or any part of them such as those described in U.S. patent application Ser. No. 10/077,446 titled “Method And System For A Range Of Automatic, Semi-Automatic, And Manual Grid Finding During Feature Extraction From Molecular Array Data”, or Ser. No. 09/589,046 “Method And System For Extracting Data From Surface Array Deposited Features”, or U.S. Pat. No. 5,721,435, all incorporated herein by reference.

Results of methods of the present invention may be used to make an assessment whether one or more targets is present in a sample to which an array was exposed, or whether an organism from which the sample was obtained exhibits a particular condition (for example, gene expression level or cancer). In certain embodiments, the results of a reading (processed or not) may be forwarded (such as by communication) to a remote location if desired, and received there for further use (such as further processing). By “remote location” is meant a location other than the location at which the sample evaluation device is present and sample evaluation occurs. For example, a remote location could be another location (e.g., office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, Internet, etc.

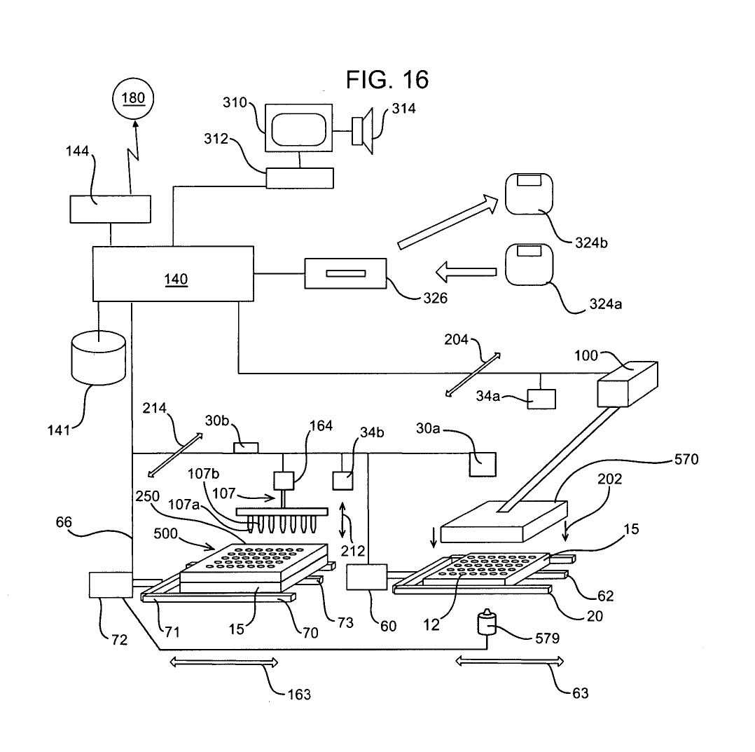

The fluid contacting methods of the subject invention may be partially or completely automated. For example, an automated system as illustrated in FIG. 16 may be employed. As such, the subject methods are amenable to high throughput applications. In automated versions of the subject methods, automated apparatuses are employed and may include a manner for precisely controlling the position of an array assembly with respect to a reservoir plate and/or an array assembly (and/or reservoir plate) with respect to a fluid contacting station and/or fluid removal station (which may or may not be the same) and/or a wash station. Embodiments include apparatuses that include at least one fluid station and which are configured to introduce fluid to, and/or remove fluid from, an array assay component, such as an array assembly/reservoir plate fluid contacting structure, positioned with respect to the fluid station, e.g., through the holes of the reservoir plate. Such embodiments may also include mechanisms for precisely positioning an array assay component, such as an array assembly/reservoir plate fluid contacting structure, with respect to a fluid station to accomplish the above-described fluid contact and/or fluid removal. An XYZ translational mechanism may be employed for such positioning.

One such automated system that may be employed in the practice of the subject methods is described with reference FIG. 16, which shows an apparatus capable of executing a method of the present invention. The apparatus is described as configured for use with an array assembly which may be an array assembly of any configuration and thus is not limited in any regard to the particular configuration of array assembly or reservoir plate. The apparatus shown essentially has two sections, a first section at which fluid is at least contacted with one or more arrays of the array assembly through holes in the reservoir plate, and a second section at which the array is washed and optionally dried. Other stations may also be present. For example, a separate station for positioning a reservoir plate relative to an array assembly may be provided. Alternatively, this may be accomplished at first fluid contacting station. Also, an optional incubation station for incubating a fluid with arrays may be provided, e.g., that may include a heater, which may be part of the first station or second station or may be a separate station. While the two sections are shown as part of one apparatus in FIG. 16, it will be appreciated that they may be entirely separate with the first section at least introducing a fluid to defined regions of an array assembly operatively positioned with respect to the first station which may then be forwarded to the wash station for washing and optionally drying, with there possibly being one or more first sections and one or more second sections remote from each other.

The first section of the apparatus of FIG. 16 includes a first fluid contacting station 70 which can retain a mounted structure that includes an array assembly 15 and a reservoir plate either spaced a capillary distance apart from the array surface of the array assembly or physically touching a surface of the array assembly. In those embodiments where the reservoir plate is physically touching a surface of the array assembly, the reservoir plate may be configured to only contact the array assembly surface about the array assembly perimeter so as not to unintentionally damage any of the arrays. For example, a border of a sealing material may be disposed about the perimeter of the reservoir plate. The first station at least introduces a fluid to defined region of a surface of the array assembly through one or more holes of the reservoir plate and may also, in certain embodiments, also remove the fluid through one or more holes of the reservoir plate. Also included is third transporter 72 of a transporter system and a fluid transfer element 107 which may include one or more individual fluid transfer elements 107a, 107b . . . such that there may be the same or similar number as the number of holes of the reservoir plate in certain embodiments.

Fluid may be delivered from fluid transfer element 107 while the fluid contacting structure 500 is advanced beneath it by third transporter 72, all under control of a processor 140. The transporter system may include a carriage 73 connected to transporter 73 controlled by processor 140. Transporter 72 moves station 70 to position an array assembly in an operative position with respect to the fluid transfer element. Transporter 72 and carriage 73 are used to execute one axis positioning of station 70 (and hence a mounted array assembly/reservoir plate structure thereon) facing the transfer element, by moving it in the direction of axis 163, while fourth transporter 164, also under control of processor 140, is used to provide adjustment of the position of transfer element 107 in a direction of axis 214. In this manner, transfer element may be moved into position to dispense a fluid such as sample fluid to arrays of the array assembly by depositing fluid in the holes of the reservoir plate. The various transporters enable relative movement of the array assembly/reservoir plate structure and the transfer element so that the transfer element may introduce fluid to certain holes at one time (e.g., a first row or column of the reservoir plate) and then be moved into another relative position to introduce fluid into other holes (e.g., a second row or column of the reservoir plate). This relative positioning may be repeated until all holes and thus all arrays of the array assembly, intended to be contacted with fluid are so contacted. Of course, the transfer element may also be configured to introduce fluid into all of the holes of a positioned reservoir plate at the simultaneously if the number of transfer elements is as least as great as the number of holes of the reservoir plate. Transfer element 107 may also optionally be moved in a vertical direction 212, by another suitable transporter (not shown). When the present application recites “positioning”, “moving”, or similar, one element (such as transfer element 107) in relation to another element (such as one of the array assembly/reservoir plate structure 500) it will be understood that any required moving can be accomplished by moving either element or a combination of both of them.

The first station may also include an optional heating element (not shown). Fluid such as sample fluid is thus in fluid communication with fluid transport element 107, e.g., may be held in a suitable reservoir connected in fluid communication with element 107 such as by tubing or the like. In-line valves and pressure sources may also be employed (not shown). Pins or similar means (not shown) may be provided on station 70 by which to approximately align structure 500 to a nominal position thereon (with optional alignment marks on the structure for more refined alignment). Station 70 may include a vacuum chuck connected to a suitable vacuum source (not shown) to retain structure 500 without exerting too much pressure thereon, since some or all of structure 500 may be made of glass. The fluid transfer element, transporter system, and processor 140 together act as a first fluid contacting station of the apparatus, e.g., to contact sample to a defined region of an array assembly through holes of a reservoir plate.

The second section of the apparatus of FIG. 16 includes wash station 20. In certain embodiments, the wash station may be configured to wash the arrays according to the embodiments of FIGS. 14A and 14B in which a fluid transfer element removes the first-deposited fluid from the structure 500 through the holes of the reservoir plate and wash fluid is flooded through the holes to wash the arrays of the array assembly. Some or all of this may be accomplished at the first station using the same fluid transfer element previously used to introduce fluid to the arrays or may be a different transfer element, e.g., at the second station or the like. In the wash embodiments of FIGS. 14A and 14B, regardless of the station at which the fluid introduced at the first station is removed from the arrays, the array assembly and/or reservoir plate, if the plate is employed for washing, may be transported to the second station using the transporter system so that a wash fluid may be flushed about the array assembly, e.g., through the holes of a reservoir plate.

In other embodiments, the wash station may be configured to wash the arrays according to the embodiments of FIGS. 15A-15C, which embodiment is shown in FIG. 16. Station 20 is provided on which an array assembly may be mounted and retained. The reservoir plate may be removed from the array assembly at station 20 or may be removed prior, e.g., at the first wash station or the like. For example, a robotic arm which may, e.g., include a vacuum chuck, may be moved into position to remove the reservoir plate a distance, if not all together, from the array assembly or the reservoir plate may be used for washing. Pins or similar means (not shown) may be provided on station 20 by which to approximately align array assembly 15 to a nominal position thereon. Station 20 may include a vacuum chuck connected to a suitable vacuum source (not shown) to retain assembly 15 without exerting too much pressure thereon, since assembly 15 may be made of glass. A fluid flood chamber 570 is provided which is configured to flood the entire surface of assembly 15 with a fluid such as wash fluid as described above. The flood chamber is positionable about assembly 15. Fluid such as wash fluid is thus in fluid communication with hood 570, e.g., may be held in a suitable wash fluid reservoir connected in fluid communication with hood 570 such as by tubing or the like. In-line valves and pressure sources may also be employed (not shown).

Station 20 includes optional drive motor 579 for rotating the assembly to dry it after exposure to the wash fluid. This may be accomplished at a separate optional drying station (not shown) downstream from the wash station.

The transporter system may include a carriage 62 connected to a first transporter 60 controlled by processor 140 through line 66, and a second transporter 100 also controlled by processor 140. Transporter 60 moves holder 20 to position an array assembly (which may or may not have a reservoir plate operatively positioned relative thereto) in an operative position with respect to the wash station. Transporter 60 and carriage 62 are used to execute one axis positioning of station 20 (and hence mounted array assembly with or without a reservoir plate) facing the wash fluid dispenser 570, by moving it in the direction of axis 63, while transporter 100 is used to provide adjustment of the position of dispenser 570 in a direction of axis 204. In this manner, dispenser 570 may be moved into position to dispense wash fluid over an array assembly and moved out of position at appropriate times, e.g., when washing and/or drying is complete. Dispenser 570 may also optionally be moved in a vertical direction 202, by another suitable transporter (not shown) and its angle of rotation with respect to dispenser 570 also adjusted. The wash dispenser, the transporter system, and processor 140 together act as the wash system of the apparatus.

In many embodiments, drying of the array assembly is also performed at station 20, where such drying is described above. For example, after washing, the array assembly is rotated using motor 570 connected to processor 140.

Encoders 30a and 30b communicate with processor 140 to provide data on the exact location of station 70 and station 20 (and hence array assembly 500 and/or reservoir plate 2509 if positioned correctly on a station), while encoders 34a and 34b provide data on the exact location of fluid transfer element 107 and wash dispenser 570, respectively. Any suitable encoder, such as an optical encoder, may be used which provides data on linear position.

Processor 140 may also have access through a communication module 144 to a communication channel 180 to communicate with a remote station. Communication channel 180 may, for example, be a Wide Area Network (“WAN”), telephone network, satellite network, or any other suitable communication channel. Array parameters such as the number of arrays, types of arrays, addresses of the arrays, and fluid parameters such as type and amount of fluid, etc. may be communicated to the processor via such a remote station. It will also be appreciated that processor 140 may be programmed from any computer readable medium carrying a suitable computer program.

The apparatus further includes a display 310, speaker 314, and operator input device 312. Operator input device 312 may, for example, be a keyboard, mouse, or the like. Processor 140 has access to a memory 141, and controls fluid transfer element 107 as well as amount of fluid to be dispensed and the like, and wash dispenser 570, operation of the transporter system, and operation of display 310 and speaker 314. Memory 141 may be any suitable device in which processor 140 can store and retrieve data, such as magnetic, optical, or solid state storage devices (including magnetic or optical disks or tape or RAM, or any other suitable device, either fixed or portable). Processor 140 may include a general purpose digital microprocessor suitably programmed from a computer readable medium carrying necessary program code, to execute all of the steps required by the present invention, or any hardware or software combination which will perform those or equivalent steps. The programming may be provided remotely to processor 141 through communication channel 180, or previously saved in a computer program product such as memory 141 or some other portable or fixed computer readable storage medium using any of those devices mentioned below in connection with memory 141. For example, a magnetic or optical disk 324a may carry the programming, and can be read by disk writer/reader 326. A separator 152 may be provided to separate assembly 15 into array. Prior to or after separation, if performed, the array assembly may then be transported to a station that includes a reader, as described above, for reading an array (not shown).

Also, as noted above one or more identifiers in the form of bar codes or the like may be attached or printed onto sections of the array assembly. If identifiers are present, they may include an indication of the location of different regions on a substrate to which arrays are present and where and/or other information that may be relevant to fluid contacting methods. They can then be read by a bar code reader (not shown) at one or more of the stations, and received by processor 140 to then control aspects of the fluid contacting. Any of the foregoing types of information on the different regions can be contained within the bar codes 356 (or other identifiers) or in a file previously linked to them. Regardless of the foregoing, at any point in the operation of the apparatus of FIG. 16, processor 140 may associate each array with an identifier such as a bar code, which identifier may carry an indication of the respective array or may be linked to a file carrying such information. The file and linkage may be stored by processor 140 and saved into memory 141 or may be written onto a portable storage medium 324b for use at a later time.

Kits

Finally, kits for use in practicing the subject invention are also provided. The subject kits may include one or more reservoir plates. Embodiments may also include one or more array assemblies. For example, embodiments may include one or more reservoir plates and one or more array assemblies for use with the reservoir plates.

The kits may further include one or more additional components necessary for carrying out an analyte detection assay, such as sample preparation reagents, buffers, labels, and the like. As such, the kits may include one or more containers such as vials or bottles, with each container containing a separate component for the assay, and reagents for carrying out an array assay such as a nucleic acid hybridization assay or the like. The kits may also include a denaturation reagent for denaturing the analyte, buffers such as hybridization buffers, wash mediums, enzyme substrates, reagents for generating a labeled target sample such as a labeled target nucleic acid sample, negative and positive controls.

In addition to one or more chemical arrays, the subject kits may also include written instructions for using the reservoir plates to contact fluid with a defined region of an array assembly, e.g., for use in array assays such as hybridization assays or protein binding assays. The instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g. via the Internet, are provided. An example of this embodiment is a kit that includes a web address where the instructions can be viewed and/or from which the instructions can be downloaded. As with the instructions, this means for obtaining the instructions is recorded on a suitable substrate.

In many embodiments of the subject kits, the components of the kit are packaged in a kit containment element to make a single, easily handled unit, where the kit containment element, e.g., box or analogous structure, may or may not be an airtight container, e.g., to further preserve the one or more chemical arrays and reagents, if present, until use.

It is evident from the above results and discussion that the above-described invention provides devices and methods for contacting a fluid with a defined region of an array assembly. Embodiments of the above-described invention may provide one or more of the advantages described above, or other advantages, and which include high throughput fluid contacting, ease of use, and use with a variety of different array configurations ranging from single arrays to sets of arrays held together on a common carrier. As such, the subject invention represents a significant contribution to the art.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A reservoir plate comprising a support comprising a plurality of holes through said support, wherein each hole is alignable with a respective chemical array of an array assembly when said reservoir plate and said array assembly are operatively positioned relative to each other.

2. The reservoir plate of claim 1, wherein said plate comprises 2n by 3n through-holes.

3. The reservoir plate of claim 2, wherein n is 4, 8 or 16.

4. The reservoir plate of claim 1, wherein said plate comprises 4x through holes.

5. The reservoir plate of claim 4, wherein x is an integer that ranges from about 1 to about 20.

6. A structure for performing an array assay, said structure comprising:
(a) an array assembly comprising at least one array; and
(b) a reservoir plate comprising a support comprising a plurality of holes through said plate, wherein each hole is alignable with a respective chemical array of an array assembly when said reservoir plate and said array assembly are operatively positioned relative to each other.

7. The structure of claim 6, wherein a surface of said reservoir plate is spaced apart from a surface of said array assembly by a capillary distance.

8. The structure of claim 7, further comprising at least one spacer element positioned between said array assembly and said reservoir plate dimensioned to provide said capillary distance.

9. The structure of claim 6, wherein said reservoir plate is contacted to a surface of said array assembly comprising said at least one array.

10. The structure of claim 6, wherein said array assembly comprises a set of chemical arrays held together by a common carrier.

11. The structure of claim 10, wherein said common carrier is a one-piece substrate having a surface on which said set of chemical arrays are disposed.

12. The structure of claim 10, wherein said common carrier comprises a substrate holder and said set of chemical arrays are mounted at different locations on said holder.

13. The structure of claim 10, wherein said set of chemical arrays are separable into multiple sub-sets of chemical arrays.

14. The structure of claim 10, wherein said structure is associated with an apparatus comprising a fluid station configured to introduce fluid through at least one hole of said reservoir plate to said array assembly when present at said fluid station.

15. The structure of claim 14, wherein said fluid comprises a sample for testing with at least one array of said array assembly or a wash fluid.

16. A method of contacting a fluid with a defined region of a surface of an array assembly comprising at least one chemical array, said method comprising:
(a) positioning a reservoir plate comprising a support comprising a plurality of holes through said plate relative to said surface such that a capillary space is provided between said reservoir plate and said surface of said array assembly; and
(b) introducing said fluid through at least one hole of said reservoir plate such that said fluid contacts a defined region of said array assembly surface.

17. The method of claim 16, wherein said method comprises maintaining said fluid at said defined region by at least one of capillary forces and a fluid barrier positioned about said defined region.

18. The method of claim 16, wherein said defined region comprises at least one chemical array and said fluid is maintained at said chemical array without cross-contamination with fluid present about any other chemical array of said array assembly.

19. The method of claim 16, wherein said method further comprises, following step (b), contacting said defined region at least one more time with the same or different fluid.

20. The method of claim 19, wherein said first contacted fluid is a sample-comprising fluid or a wash fluid and said second contacted fluid is a sample-comprising fluid or a wash fluid.

21. The method of claim 19, wherein said contacting said defined region comprises flushing said surface of said array assembly.

22. The method of claim 21, wherein said flushing comprises a reservoir plate and said fluid is introduced to said array assembly through at least one hole of said reservoir plate.

23. The method of claim 21, wherein said flushing does not comprises a reservoir plate.

24. The method of claim 16, wherein said array assembly comprises a set of chemical arrays held together by a common carrier.

25. The method of claim 24, wherein said common carrier is a one-piece substrate having a surface on which said set of chemical arrays are disposed or said common carrier comprises a substrate holder and said set of chemical arrays are mounted at different locations on said holder.

26. The method of claim 16, further comprising separating said set of chemical arrays into multiple sub-sets of chemical arrays, each carried on a separate substrate.

27. The method of claim 16, wherein said fluid is sample and said method further comprises:
maintaining said sample at said defined region under conditions sufficient to perform an array assay;
removing said sample from said array assembly surface; and
detecting the presence of any binding complexes on said surface.

28. A method comprising forwarding data representing a result of a reading obtained by the method of claim 27.

29. The method according to claim 28, wherein said data is transmitted to a remote location.

30. A method comprising receiving data representing a result of a reading obtained by the method of claim 27.

31. A method of contacting a fluid with a defined region of a surface of an array assembly, said method comprising:
(a) operatively positioning a reservoir plate, comprising a support comprising a plurality of holes through said plate, relative to said surface of said array assembly;
(b) introducing said fluid through at least one hole of said reservoir plate such that said fluid contacts a defined region of said array assembly surface;
(c) removing said reservoir plate a distance from said array assembly to flood a surface of said array assembly with a liquid; and
(d) flooding a surface of said array assembly with a liquid.

32. The method of claim 31, wherein said positioning comprises spacing a surface of said reservoir plate a capillary distance from a surface of said array assembly.

33. The method of claim 31, wherein said positioning comprises contacting a surface of said reservoir plate with a surface of said array assembly.
